(19) United States
(12) Patent Application Publication
(10) Pub. No.: US 2026/0053529 A1
(43) Pub. Date: Feb. 26, 2026

(54) CANNULA SYSTEM AND METHOD FOR TISSUE REPAIR

(71) Applicant: Arthrex, Inc., Naples, FL (US)

(72) Inventors: Stephan Bosch, Naples, FL (US); John Seyler, Naples, FL (US); Patrick J. Denard, Medford, OR (US); Grant H. Garcia, Mercer Island, WA (US)

(73) Assignee: Arthrex, Inc., Naples, FL (US)

(21) Appl. No.: 19/303,659

(22) Filed: Aug. 19, 2025

Related U.S. Application Data
(60) Provisional application No. 63/685,524, filed on Aug. 21, 2024.

(51) Int. Cl. A61B 17/34; A61B 17/04; A61B 17/17; A61F 2/08

(52) U.S. Cl. CPC A61B 17/3468; A61B 17/0401; A61B 17/1796; A61B 17/3423; A61B 17/3472; A61B 17/3498; A61F 2/0811; A61B 2017/0409; A61B 2017/0464; A61F 2002/0852; A61F 2002/0882

(57) ABSTRACT

A cannula system and method for tissue repair includes a cannula defining a central lumen and a divider removably coupled to the cannula. The divider has a divider shaft to separate the central lumen into at least two discrete working channels.

### CROSS-REFERENCE TO RELATED APPLICATION

This application priority to and the benefit of U.S. Provisional Application 63/685,524 filed on Aug. 21, 2024. The disclosure of the above application is incorporated herein by reference.

### BACKGROUND

The present disclosure relates to medical devices and methods for tissue repair, and more particularly relates to a cannula system and an associated method for soft tissue repair, such as a remplissage.

Surgery may be required to reattach the soft tissue to the bone to promote healing of the soft tissue and corresponding joint(s). In certain instances, the surgery may be performed arthroscopically, using small incisions to access a region within an anatomy of a patient proximate the corresponding joint(s).

### SUMMARY

This section provides a general summary of the disclosure and is not a comprehensive disclosure of its full scope or all of its features.

The present disclosure provides a cannula system, which enables an associated method for the repair of soft tissue. The cannula system may generally include a cannula with a central lumen that may enable various medical devices to be inserted into the anatomy to repair the soft tissue.

In one example, the cannula system may include the cannula and an obturator, which may be received in the central lumen to direct the cannula percutaneously into the anatomy. The cannula may be oval in cross-section, which assists in coupling the cannula to the anatomy. The cannula system may also include a divider, which may divide the central lumen of the cannula into at least two substantially independent, discrete or separate working channels. In one example, the cannula includes a cap, which may receive a portion of the divider to couple the divider to the cannula in a single orientation. The cap may also include one or more suture retaining slots or suture cleats. The cannula may include a valve, which restricts the flow of fluids through the cannula. The cannula system may be used to perform a surgical procedure to repair soft tissue, such as a remplissage.

In one example, a cannula system is provided that may include a cannula defining a central lumen and a divider removably coupled to the cannula. The divider may have a shaft configured to separate the central lumen into at least two working channels.

A method may be provided for soft tissue repair, which may include inserting a cannula into an incision in an anatomy. The cannula may define a central lumen and include a cannula tube that may have an elliptical cross-section. The method may include orientating the cannula tube such that a major axis of the cannula tube is substantially perpendicular to the incision and inserting a divider into the central lumen to separate the central lumen into at least two working channels. The method may include inserting a soft anchor into one of the at least two working channels to repair soft tissue.

Also provided is a cannula system that includes a cannula defining a central lumen and a divider removably coupled to the cannula. The divider has a divider shaft configured to separate the central lumen into at least two discrete working channels.

The central lumen is elliptical in cross-section. The cannula includes a valve received within the central lumen, and the divider shaft extends through the valve. The cannula includes a cap, and the cap defines at least one suture retaining slot. The at least one suture retaining slot defines a tortuous path configured to retain at least one flexible strand. The cap defines a coupling groove configured to receive a portion of the divider, and the coupling groove is spaced apart from the at least one suture retaining slot about a perimeter of the cap. The cap defines a central cap bore configured to be in communication with the central lumen. The at least one suture retaining slot defines a slit configured to retain at least one flexible strand. The cannula system includes an obturator configured to be removably coupled to the central lumen. An obturator shaft of the obturator has an elliptical cross-section. A center of a first one of the at least two working channels is spaced apart from a second center of a second one of the at least two working channels by at least one millimeter. The divider shaft includes at least two side channels and each of the at least two side channels cooperate with a sidewall of the central lumen to define the at least two working channels.

A method for soft tissue repair includes inserting a cannula into an incision in an anatomy, with the cannula defining a central lumen. The method includes inserting a divider into the central lumen to separate the central lumen into at least two discrete working channels, and inserting a soft anchor into one of the at least two working channels to repair soft tissue.

The central lumen has an elliptical cross-section, and the method includes orientating the cannula such that a major axis of the cannula is substantially perpendicular to the incision. The method includes inserting an obturator having an obturator shaft with an elliptical cross-section through the central lumen and directing the cannula through the anatomy with the obturator. The method includes inserting a drill guide into the one of the at least two working channels and inserting a drill through the drill guide to form a bone socket prior to inserting the soft anchor. The method includes tensioning the soft anchor to set the soft anchor within the bone socket, coupling at least one flexible strand associated with the soft anchor to a suture retaining slot defined in the cannula to couple the at least one flexible strand to the cannula, inserting the drill guide into a second one of the at least two working channels and inserting the drill through the drill guide and the second one of the at least two working channels to form a second bone socket without contacting the at least one flexible strand. The method includes inserting a second soft anchor into the second one of the at least two working channels, tensioning the soft anchor to set the soft anchor within the second bone socket, and coupling at least one second flexible strand associated with the second soft anchor to a second suture retaining slot defined in the cannula to couple the at least one second flexible strand to the cannula. The method includes coupling the at least one flexible strand of the soft anchor to the second soft anchor, coupling the at least one second flexible strand of the second soft anchor to the soft anchor, removing the divider from the cannula and tensioning the at least one flexible strand and the at least one second flexible strand to repair the soft tissue. The method includes removing the cannula from the anatomy after the tensioning the at least one flexible strand and at least one second flexible strand.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 is a perspective partially exploded view of a kit including an exemplary cannula system for soft tissue repair in accordance with the various teachings of the present disclosure.

FIG. 2 is a perspective view of an obturator coupled to a cannula of the cannula system.

FIG. 3 is a side view of the obturator, in which a major axis of an obturator shaft is shown.

FIG. 4 is a side view of the obturator, in which a minor axis of the obturator shaft is shown.

FIG. 5 is a cross-sectional view of the obturator shaft taken along line 5-5 of FIG. 3.

FIG. 6 is an exploded view of the cannula system.

FIG. 7 is a top end view of a cannula cap associated with the cannula system.

FIG. 8 is a cross-sectional view of the cannula cap taken along line 8 of FIG. 7.

FIG. 9 is a cross-sectional view of the cannula cap taken along line 8 of FIG. 7.

FIG. 10 is a cross-sectional view of the cannula cap taken along line 10-10 of FIG. 7.

FIG. 11 is a bottom end view of the cannula cap.

FIG. 12 is a side view of the cannula cap.

FIG. 13 is another side view of the cannula cap.

FIG. 14 is a top end view of a valve associated with the cannula.

FIG. 15 is a side view of the valve.

FIG. 16 is a side view of a cannula body associated with the cannula.

FIG. 17 is a cross-sectional view of the cannula body taken along line 17-17 of FIG. 16.

FIG. 18 is a top end view of the cannula body.

FIG. 19 is a bottom end view of the cannula body.

FIG. 20 is a side view of a divider associated with the cannula system.

FIG. 21 is a cross-sectional view of the divider taken along line 21-21 of FIG. 20.

FIG. 22 is a top end view of the divider.

FIG. 23 is a bottom end view of the divider.

FIG. 24 is a detail view of the divider taken at 24 on FIG. 23.

FIG. 25 is a perspective view of the divider coupled to the cannula of the cannula system.

FIG. 26 is a cross-sectional view of the divider and the cannula taken along line 26-26 of FIG. 25.

FIG. 27 is a cross-sectional view of the divider and the cannula taken along line 27-27 of FIG. 25.

FIG. 28 is a bottom end view of the divider coupled to the cannula.

FIG. 29 is a top end view of the divider coupled to the cannula.

FIG. 30 is a side view of the cannula.

FIG. 31 is a cross-sectional view of the cannula taken along line 31 of FIG. 30.

FIG. 32 is a top end view of the cannula.

FIG. 33 is a cross-sectional view of the obturator and the cannula taken along line 33-33 of FIG. 2.

FIGS. 34-44 illustrate an exemplary method for soft tissue repair with the cannula system.

FIG. 45 is a bottom end view of another exemplary divider coupled to the cannula.

FIG. 46 is a bottom end view of another exemplary divider coupled to the cannula.

FIG. 47 is a bottom end view of another exemplary divider coupled to the cannula.

FIG. 48 is a perspective partially exploded view of a kit including an exemplary cannula system for soft tissue repair in accordance with the various teachings of the present disclosure.

FIG. 49 is a side view of an obturator coupled to a cannula of the cannula system.

FIG. 50 is a top view of the obturator.

FIG. 51 is a cross-sectional view of the obturator, taken along line 51 of FIG. 50.

FIG. 52 is a side view of the obturator.

FIG. 53 is a rear perspective view of a cannula cap associated with the cannula system of FIG. 48, which includes a cannula cap insert.

FIG. 54 is a top view of the cannula cap in which the cannula cap insert is removed from the cannula cap.

FIG. 55 is a top view of the cannula cap including the cannula cap insert.

FIG. 56 is a cross-sectional view of the cannula cap taken along line 56-56 of FIG. 55.

FIG. 57 is a cross-sectional view of the cannula cap taken along line 57-57 of FIG. 55.

FIG. 58 is a bottom view of the cannula cap.

FIG. 59 is a side view of the cannula cap including the cannula cap insert.

FIG. 60 is another side view of the cannula cap.

FIG. 61 is a cross-sectional view of a cannula associated with the cannula system taken along line 61-61 of FIG. 48.

FIG. 62 is a top view of the cannula.

FIG. 63 is a top view of a divider associated with the cannula system of FIG. 48.

FIG. 64 is a side view of the divider.

FIG. 65 is a cross-sectional view of the divider taken along line 65-65 of FIG. 64.

FIG. 66 is a bottom view of the divider.

FIG. 67 is bottom end view of the divider coupled to the cannula.

FIG. 68 is a cross-sectional view of the divider coupled to the cannula.

FIG. 69 is a cross-sectional view of the obturator coupled to the cannula, taken along line 69-69 of FIG. 49.

FIGS. 70-76 illustrate an exemplary method for soft tissue repair with the cannula system of FIG. 48.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features. In addition, there is no intention to be bound by any expressed or implied theory presented in the preceding technical field, background, brief summary or the following detailed description. Further, the present disclosure may be practiced in conjunction with any method for soft tissue repair that would benefit from a cannula with at least two working channels, and the use of the cannula system to perform a remplissage soft tissue repair is merely one example according to the present disclosure. Further, it should be noted that many alternative or additional functional relationships or physical connections may be present in an example of the present disclosure. In addition, while the figures shown herein depict an example with certain arrangements of elements, additional intervening elements, devices, features, or components may be present. It should also be understood that the drawings are merely illustrative and may not be drawn to scale.

As used herein, the term “axial” refers to a direction that is generally parallel to or coincident with an axis of rotation, axis of symmetry, or centerline of a component or components. For example, in a cylinder or disc with a centerline and generally circular ends or opposing faces, the “axial” direction may refer to the direction that generally extends in parallel to the centerline between the opposite ends or faces. In certain instances, the term “axial” may be utilized with respect to components that are not cylindrical (or otherwise radially symmetric). For example, the “axial” direction for a rectangular housing containing a rotating shaft may be viewed as a direction that is generally parallel to or coincident with the rotational axis of the shaft. Furthermore, the term “radially” as used herein may refer to a direction or a relationship of components with respect to a line extending outward from a shared centerline, axis, or similar reference, for example in a plane of a cylinder or disc that is perpendicular to the centerline or axis. In certain instances, components may be viewed as “radially” aligned even though one or both of the components may not be cylindrical (or otherwise radially symmetric). Furthermore, the terms “axial” and “radial” (and any derivatives) may encompass directional relationships that are other than precisely aligned with (e.g., oblique to) the true axial and radial dimensions, provided the relationship is predominantly in the respective nominal axial or radial direction. As used herein, the term “about” denotes within 15% to account for manufacturing tolerances. In addition, the term “substantially” denotes within 15% to account for manufacturing tolerances.

The present disclosure provides a cannula system, which can be used in an orthopedic surgical procedure involving the fixation of soft tissue to bone. The cannula system may generally include a cannula that defines a central lumen. An obturator may be received through the central lumen to percutaneously insert the cannula into an anatomy at a predetermined site for soft tissue repair, such as proximate an infraspinatus tendon. The cannula system may also include a divider, which may be coupled to the cannula to divide the central lumen into at least two discrete working channels. By providing at least two substantially discrete and separate working channels, sutures associated with the soft tissue repair may be protected from other medical instruments and devices used during the surgical procedure, such as drill bits, etc. In addition, the at least two separate working channels may inhibit the sutures associated with the surgical procedure from becoming tangled or twisted together during the surgical procedure.

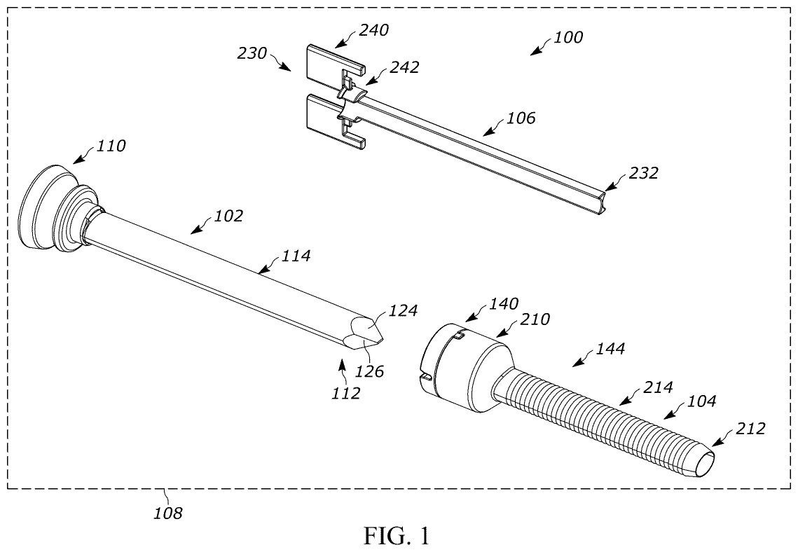

With reference to FIG. 1, a cannula system 100 is shown. In one example, the cannula system 100 may include an obturator 102, a cannula 104 and a divider 106. As will be described, the cannula system 100 enables the percutaneous insertion of the cannula 104 into the anatomy for performing a soft tissue repair surgical procedure, such as a remplissage. Generally, the cannula system 100 may enable a surgeon to perform the soft tissue repair without a line of sight or “blind”, while ensuring that one or more flexible strands or sutures involved in the soft tissue repair are protected during the surgical procedure. The cannula system 100 may also permit the surgeon to perform other portions of the soft tissue repair procedure while retaining tension on the flexible strands or sutures so that the final tensioning of the soft tissue may be completed near the end of the surgical procedure. It should be noted that while the cannula system 100 may be described herein as being using with a remplissage, the cannula system 100 may be used with any suitable surgical procedure, including, but not limited to Hill-Sach's repairs, transtendon repairs (for example, Partial Articular Surface Tendon Avulsion (PASTA) lesion repairs), fixation of soft tissue (ligament, tendon, graft, etc.) to bone, and for any soft tissue repairs including PASTA, labral, rotator cuff, Achilles tendon, biceps and hip repairs, etc. The cannula system 100, the obturator 102, the cannula 104 and the divider 106 may be packaged together in a kit 108, for example.

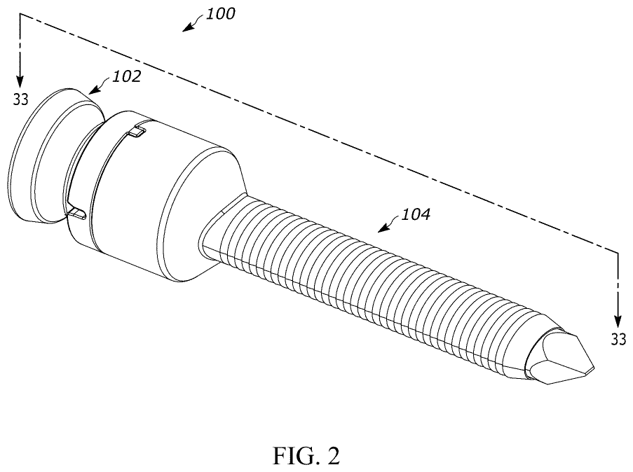

Generally, the obturator 102 may assist in the insertion of the cannula 104 into the anatomy. In one example, the obturator 102 may include a proximal obturator end 110, a distal obturator end 112 opposite the proximal obturator end 110, and an obturator shaft 114 that interconnects the proximal obturator end 110 with the distal obturator end 112. The obturator 102 may be composed of a biocompatible material, for example, a biocompatible polymer-based material, including, but not limited to polycarbonate. The obturator 102 may be manufactured through any suitable technique, including, but not limited to additive manufacturing. Generally, the obturator 102 may be clear or transparent, but may comprise a color or color markings. The obturator 102 may be described and illustrated herein as comprising an integral, monolithic or one-piece component, however, in other examples, the proximal obturator end 110 may be separately formed and coupled to the obturator shaft 114 via ultrasonic welding, for example. With reference to FIG. 2, the proximal obturator end 110 extends outwardly from the cannula 104 and may include a graspable portion 116. The graspable portion 116 enables the surgeon to control the obturator 102 and manipulate the obturator 102 to direct the cannula 104 to the predetermined portion of the anatomy.

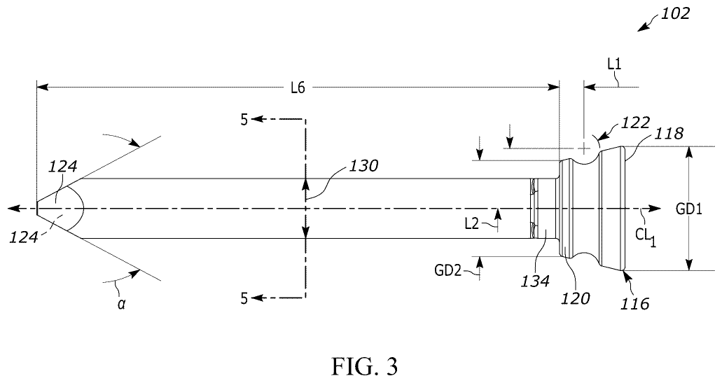

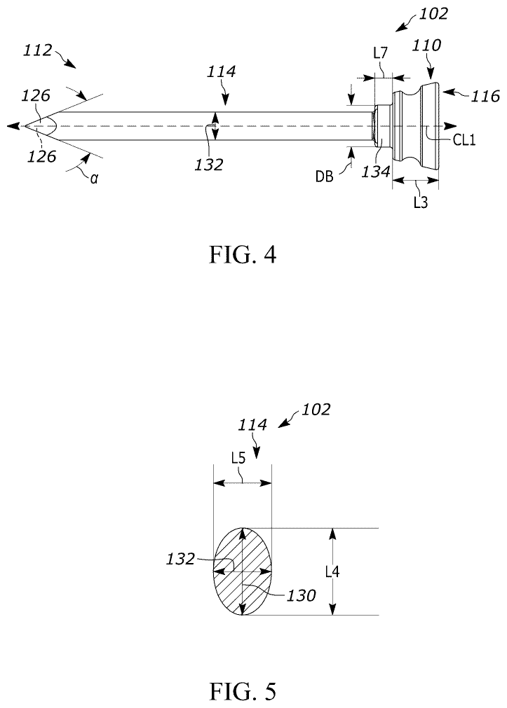

In this example, the graspable portion 116 may be circular and knob-like, however, the graspable portion 116 may have any desired shape to provide a graspable surface or manipulatable portion for use by the surgeon. With reference to FIG. 3, the graspable portion 116 may have a first diameter GD1 at a proximalmost end 118, which may be different and greater than a second diameter GD2 of the graspable portion 116 at an end 120 proximate or at the obturator shaft 114. In one example, the first diameter GD1 may be about 0.7 inches to about 1.1 inches, and the second diameter GD2 may be about 0.4 inches to about 0.9 inches. A fillet 122 may be defined between the proximalmost end 118 and the end 120 to define a radius between the two diameters GD1, GD2. The fillet 122 may have a diameter of about 0.1 inches to about 0.4 inches. The fillet 122 may be defined a first length L1 from the end 120, and in one example, the first length L1 may be about 0.08 inches to about 0.3 inches. The fillet 122 may be spaced radially outward from a centerline CL1 of the obturator 102 by a second length L2, and the second length L2 may be about 0.20 inches to about 0.60 inches. The fillet 122 along with the step between the diameters GD1, GD2 may enable the surgeon to position one or more fingers about the graspable portion 116 to manipulate the graspable portion 116 during the surgical procedure. With reference to FIG. 4, the proximal obturator end 110 extends for a third length L3, which may be about 0.4 inches to about 0.6 inches.

With reference to FIGS. 3 and 4, the distal obturator end 112 may be tapered to assist in the dilation of tissue as the obturator 102 and the cannula 104 are directed through the anatomy. In one example, the distal obturator end 112 may have a pair of first angled surfaces 124 (FIG. 3) and a pair of second angled surfaces 126 (FIG. 4). The first angled surfaces 124 may be defined on opposed sides of the obturator 102, and with reference to FIG. 4, the second angled surfaces 126 may be defined on opposed sides of the obturator 102. In one example, with reference to FIG. 3, the second angled surfaces 126 may be defined at an angle α of about 51 degrees to about 61 degrees. The angle α may be defined between a reference line that extends along the respective second angled surface 126 and the centerline CL1. In one example, with reference to FIG. 4, the first angled surfaces 124 may be defined at an angle β of about 39 degrees to about 49 degrees. The angle β may be defined between a reference line that extends along the respective first angled surface 124 and the centerline CL1. Thus, the angle α may be different and greater than the angle β.

Generally, with reference to FIG. 5, the obturator shaft 114 of the obturator 102 may be elliptical or oval in cross-section to enable the obturator shaft 114 to be positioned within the cannula 104. The obturator shaft 114 may have a major axis 130 that extends perpendicular to a minor axis 132. In one example, the major axis 130 may have a fourth length L4, which may be about 0.3 inches to about 0.5 inches, and the minor axis 132 may have a fifth length L5, which may be about 0.2 inches to about 0.4 inches. Generally, the fourth length L4 and the fifth length L5 may be predetermined such that the fourth length L4 may be different and greater than the fifth length L5 to arrive at the elliptical shape. The elliptical shape of the obturator 102 results in the first angled surfaces 124 extending at a different angle than the second angled surfaces 126. With reference back to FIG. 3, the first angled surfaces 124 may be defined along surfaces of the obturator 102 that extend substantially parallel to the major axis 130, and with reference to FIG. 4, the second angled surfaces 126 may be defined along surfaces of the obturator 102 that extend substantially parallel to the minor axis 132.

With reference to FIGS. 3 and 4, the obturator shaft 114 extends from the distal obturator end 112 to the graspable portion 116 for a sixth length L6. The sixth length L6 may be about 3.5 inches to about 4.5 inches. In one example, the obturator shaft 114 may be coupled to the graspable portion 116 via a collar 134. The collar 134 may have a third diameter D3 (FIG. 4), which may be about 0.3 inches to about 0.5 inches. Generally, the third diameter D3 may be different and less than the second diameter GD2 and the first diameter GD1. The collar 134 may have a seventh length L7, which may be about 0.1 inches to 0.3 inches. The collar 134 may assist in coupling the cannula 104 to the obturator 102 and may act as a stop that inhibits the advancement of the cannula 104 toward the graspable portion 116 along the obturator shaft 114. As will be described, in one example, the collar 134 may be received within a portion of the cannula 104 to couple the cannula 104 to the obturator 102.

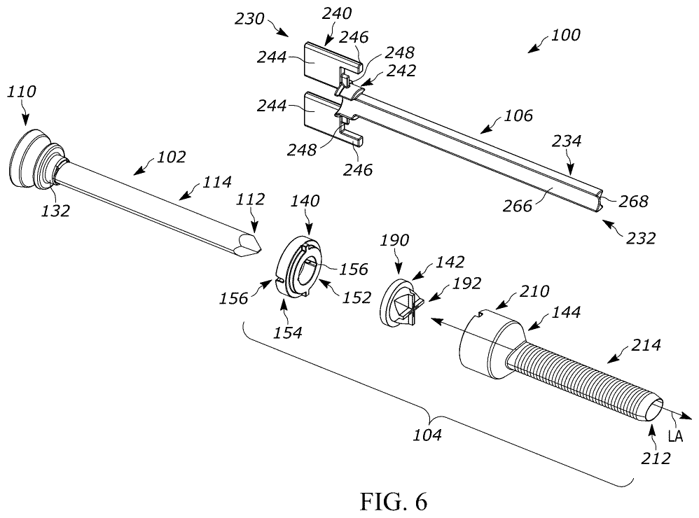

With reference to FIG. 6, an exploded view of the cannula system 100 is shown. In this example, the cannula 104 may include a cap or cannula cap 140, a dam or valve 142 and a cannula body 144. The cannula cap 140 may be composed of a biocompatible material, for example, a biocompatible polymer-based material, including, but not limited to polycarbonate. The cannula cap 140 may be manufactured through any suitable technique, including, but not limited to additive manufacturing. The cannula cap 140 may have a first cap end 150 opposite a second cap end 152, a cap sidewall 154 that interconnects the first cap end 150 and the second cap end 152, and a central cap bore 156. The cannula cap 140 may be substantially circular.

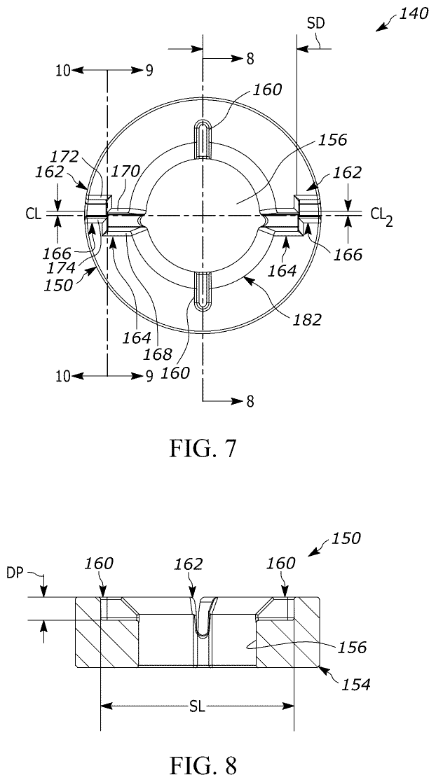

With reference to FIG. 7, a top view of the first cap end 150 is shown. The first cap end 150 may have a diameter, which may be about 0.75 inches to about 1.1 inches. Generally, the first cap end 150 may be sized and shaped to be received within and coupled to the cannula body 144. The first cap end 150 may include a pair of opposed coupling grooves 160 and a pair of opposed suture retaining slots or suture cleats 162. In this example, the coupling grooves 160 and the suture cleats 162 may be spaced apart about a perimeter or circumference of the first cap end 150. The coupling grooves 160 may be defined so as to be in communication with the central cap bore 156 and to extend from the central cap bore 156 radially outward toward the perimeter of the first cap end 150. The coupling grooves 160 may be sized and shaped to receive a portion of the divider 106 to couple the divider 106 to the cannula 104. Generally, the coupling grooves 160 may be defined to extend along an axis that may be perpendicular to a longitudinal axis LA of the cannula 104 (FIG. 6), and that may be perpendicular to an axis along which at least a portion of the suture cleats 162 are defined to extend. In one example, the opposed coupling grooves 160 each may have a width W, which may be about 0.025 inches to about 0.075 inches. With reference to FIG. 8, each of the coupling grooves 160 may be defined to extend for a depth DP into the first cap end 150, and in one example, the depth DP may be about 0.05 inches to about 1.5 inches. The coupling grooves 160 may also cooperate to define a slot having a slot length SL along the first cap end 150 from one coupling groove 160 to the opposite coupling groove 160, which may be about 0.5 inches to about 0.85 inches. The slot may receive the portion of the divider 106 to couple the divider 106 to the cannula 104.

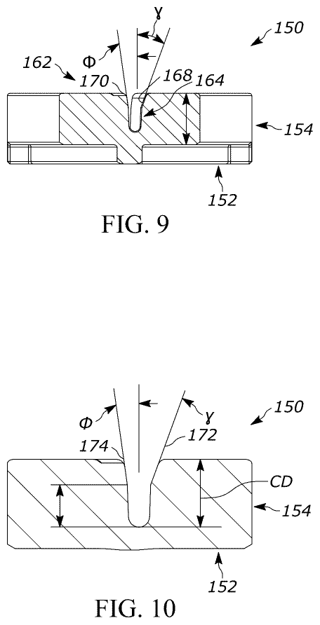

With reference back to FIG. 7, the suture cleats 162 may receive and retain sutures associated with the surgical procedure during the surgical procedure. Each of the suture cleats 162 may include a first cleat portion 164 and a second cleat portion 166. The first cleat portion 164 may be in communication with the central cap bore 156 and may extend radially from the central cap bore 156 to the second cleat portion 166. The second cleat portion 166 may be offset from, but in communication with the first cleat portion 164 to define a tortuous path that assists in retaining the flexible strands or sutures within the suture cleats 162. In one example, the second cleat portion 166 may be defined to be spaced apart from a center line of the cannula cap 140 by a spacing distance SD, which may be about 0.27 inches to about 0.46 inches. The first cleat portion 164 may be defined along a centerline CL2 of the cannula cap 140, and the second cleat portion 166 may be defined so as to be offset from the centerline CL2. The second cleat portion 166 may be in communication with the cap sidewall 154 to direct excess flexible strands or sutures to a side of the cannula 104. With reference to FIG. 9, the first cleat portion 164 may have a first cleat surface 168 that extends at an angle γ. The angle γ may be defined between the first cleat surface 168 and a reference line that extends through a center of the first cleat portion 164. A second cleat surface 170 may be opposite the first cleat surface 168 and extends at an angle φ. The angle φ may be defined between the second cleat surface 170 and the reference line that extends through the center of the first cleat portion 164. In one example, the angle φ may be different and less than the angle γ. The angle φ may be about 4 degrees to 12 degrees, and the angle γ may be about 16 degrees to about 24 degrees.

With reference to FIG. 10, the second cleat portion 166 may have a third cleat surface 172 that extends at the angle γ. The angle γ may be defined between the third cleat surface 172 and a reference line that extends through a center of the second cleat portion 166. A fourth cleat surface 174 may be opposite the third cleat surface 172 and may extend at the angle φ. The angle φ may be defined between the fourth cleat surface 174 and the reference line that extends through the center of the second cleat portion 166. The first cleat portion 164 and the second cleat portion 166 may extend for a cleat depth CD, which may be about 0.1 inches to about 0.2 inches. With reference back to FIG. 7, the third cleat surface 172 may be opposite the first cleat surface 168 along the tortuous path defined by the suture cleats 162, and the second cleat surface 170 may be opposite the fourth cleat surface 174. This may assist in retaining the flexible strands or sutures on the respective suture cleats 162. A bottom surface of each of the first cleat portion 164 and the second cleat portion 166 may have a radius or curvature, as shown in FIGS. 9 and 10.

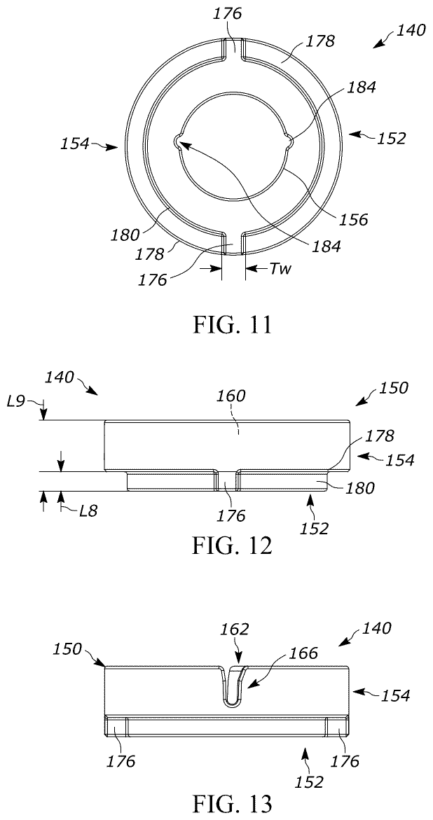

With reference to FIG. 11, the second cap end 152 may be coupled to the cannula body 144. In one example, the second cap end 152 includes a pair of coupling tabs 176, which may be opposite each other about a perimeter or circumference of the second cap end 152. Generally, the coupling tabs 176 may be aligned with the coupling grooves 160 (FIG. 12) and offset from the suture cleats 162 defined on the first cap end 150 (FIG. 13). In this example, the second cap end 152 may define a pair of relief grooves 178, which enable the second cap end 152 to be received within the cannula body 144. The relief grooves 178 may reduce an external diameter of the cap sidewall 154 at the second cap end 152 to define a coupling surface 180. The coupling tabs 176 may extend from the coupling surface 180 to an external diameter of the remainder of the cap sidewall 154. The coupling tabs 176 may be sized and shaped to be received within a portion of the cannula body 144 to assist in locating the cannula cap 140 on the cannula body 144 during assembly. The coupling tabs 176 may have a tab width TW, which may be about 0.05 inches to about 1.3 inches.

With reference to FIGS. 12 and 13, the cap sidewall 154 interconnects the first cap end 150 with the second cap end 152. The cap sidewall 154 may include the relief grooves 178 at the second cap end 152, which may extend for an eighth length L8. The eighth length L8 may be about 0.05 inches to about 1.1 inches. The cap sidewall 154 may generally extend for a ninth length L9, which may be about 0.22 inches to about 0.33 inches. The second cleat portion 166 of each of the suture cleats 162 may be defined through a portion of the cap sidewall 154.

The central cap bore 156 may be defined to extend from the first cap end 150 to the second cap end 152. With reference back to FIG. 7, the central cap bore 156 may have a diameter CD of about 0.25 inches to 0.55 inches. Generally, the central cap bore 156 may be sized to enable instruments associated with the surgical procedure to be inserted through the central cap bore 156 and into the cannula body 144. The central cap bore 156 may also include a chamfer 182 at the first cap end 150 about the central cap bore 156, which may be coupled to and contact the collar 134 of the obturator 102 when the obturator 102 may be coupled to the cannula 104. With reference to FIG. 11, the central cap bore 156 may also include a pair of opposed channels 184, which may be in communication with a respective one of the suture cleats 162. The channels 184 may be defined along the central cap bore 156 from the second cap end 152 to the respective suture cleat 162 and may assist in guiding the flexible strands or sutures into the suture cleats 162.

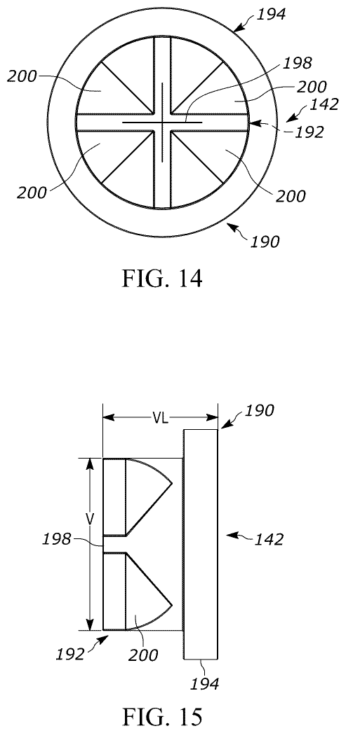

With reference back to FIG. 6, the valve 142 inhibits the flow of fluids out of the cannula body 144 when the cannula body 144 is positioned within the anatomy. The valve 142 may be composed of a biocompatible material, for example, a biocompatible polymer-based material. The valve 142 may be manufactured through any suitable technique, including, but not limited to injection molding. The valve 142 may include a first valve end 190 opposite a second valve end 192. In one example, with reference to FIG. 14, the first valve end 190 may include a sealing ring 194 and a valve opening 196. The sealing ring 194 may couple the valve 142 within the cannula body 144 and forms a seal against an interior of the cannula body 144. The valve opening 196 may extend from the first valve end 190 to the second valve end 192. In this example, the second valve end 192 may have a cross-slit 198. In one example, the valve 142 may be formed such that the cross-slit 198 results in a plurality of triangular shaped sections or leaves 200. The cross-slit 198 enables the plurality of triangular shaped leaves 200 to move from a closed position (shown in FIG. 14) to an opened position upon the insertion of an instrument or medical device through the valve 142. It should be noted that the valve 142 may be configured differently, if desired, to restrict the flow of fluids from the cannula body 144 during the surgical procedure. With reference to FIG. 15, the valve 142 may have a valve length VL of about 0.27 inches to about 0.42 inches, and a valve diameter V at the second valve end 192 of about 0.4 inches to about 0.67 inches. The sealing ring 194 may have a diameter of about 0.6 inches to about 0.8 inches, and thus, the diameter of the sealing ring 194 may be different and greater than the valve diameter V at the second valve end 192.

With reference back to FIG. 6, the valve 142 may be received within the cannula body 144. The cannula body 144 may be composed of a biocompatible material, for example, a biocompatible polymer-based material, including, but not limited to polycarbonate. The cannula body 144 may be manufactured through any suitable technique, including, but not limited to additive manufacturing. The cannula body 144 may include a first cannula end 210 opposite a second cannula end 212 and a cannula tube 214 that interconnects the first cannula end 210 with the second cannula end 212. The cannula body 144 may also define a central lumen 216 (FIG. 17), which extends from the first cannula end 210 to the second cannula end 212. The cannula body 144 may have a length BL of about 3.3 inches to about 3.6 inches.

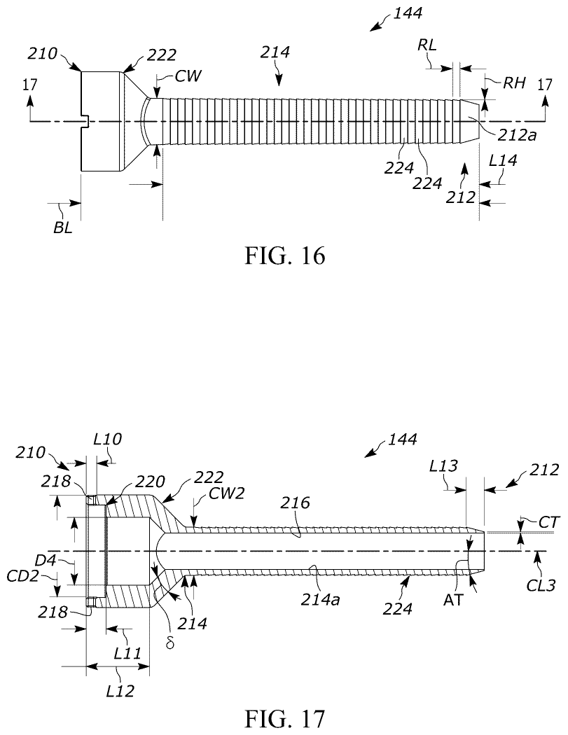

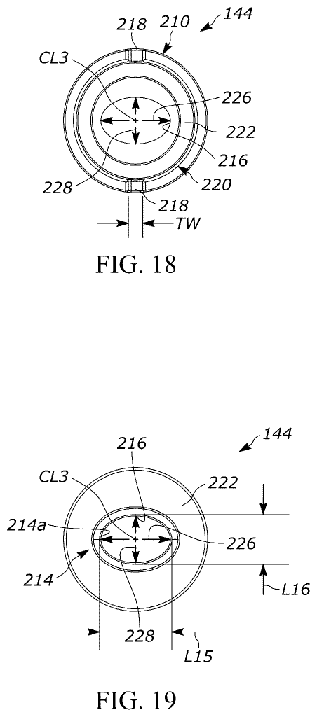

With reference to FIG. 16, the first cannula end 210 may be substantially cup-shaped and may be sized and shaped to be coupled to the cannula cap 140 and the valve 142. With reference to FIGS. 17 and 18, the first cannula end 210 may include a pair of opposed coupling slots 218, a receiving channel 220 and a tapered portion 222. The coupling slots 218 may be defined through an exterior surface of the cannula body 144 at the first cannula end 210 and may be in communication with the central lumen 216 to receive the coupling tabs 176 of the cannula cap 140. The coupling slots 218 may be sized and shaped to receive the coupling tabs 176, and in one example, are substantially rectangular. The coupling slots 218 may have a tab width TW of about 0.05 inches to 0.15 inches. With reference to FIG. 17, the coupling slots 218 may extend for a tenth length L10, which may be about 0.05 inches to about 0.11 inches.

The receiving channel 220 may be defined about the central lumen 216 and may be in communication with the opposed coupling slots 218. The receiving channel 220 receives the sealing ring 194 of the valve 142 to couple the valve 142 to the cannula body 144 along with the coupling surface 180 of the cannula cap 140. The receiving channel 220 may have a diameter CD2 of about 0.5 inches to about 1.0 inches and may have a channel length L11 of about 0.14 inches to 0.21 inches. Between the receiving channel 220 and the tapered portion 222, the central lumen 216 may have a diameter D4 of about 0.4 inches to 0.7 inches.

The tapered portion 222 may be defined proximate the cannula tube 214 and transitions the first cannula end 210 to the cannula tube 214. The tapered portion 222 may be defined so as to be spaced apart from the first cannula end 210 by a twelfth length L12, which may be about 0.3 inches to 0.6 inches. In one example, the tapered portion 222 may be defined to have a slope with an angle δ of about 40 degrees to about 50 degrees. The angle δ may be defined between a sidewall of the central lumen 216 at the receiving channel 220 and a centerline CL3 of the cannula body 144.

The second cannula end 212 tapers to form a distalmost end of the cannula 104. A taper 212a at the second cannula end 212 may extend for a thirteenth length L13 of about 0.14 inches to 0.17 inches. In one example, the taper may extend at an angle AT of about 10 degrees to 20 degrees. The angle AT may be defined between the centerline CL3 of the cannula body 144 and the exterior surface of the second cannula end 212. The second cannula end 212 may have a thickness CT at the distalmost end of about 0.005 inches to 0.015 inches.

The cannula tube 214 may extend from the first cannula end 210 to the second cannula end 212 and may have an interior surface 214a that surrounds a portion of the central lumen 216. In one example, with reference back to FIG. 16, the cannula tube 214 may include at least one or a plurality of coupling ribs 224. The plurality of coupling ribs 224 may be defined along an exterior of the cannula body 144 and may extend from the tapered portion 222 to the taper at the second cannula end 212. The plurality of coupling ribs 224 assist in retaining the cannula body 144 within the anatomy by engaging with the surrounding tissue. In one example, the plurality of coupling ribs 224 may be shaped to form a fir-tree coupling structure, with each coupling rib 224 having a rib length RL of about 0.04 inches to 0.08 inches. Each coupling rib 224 may have a rib height RH of about 0.005 inches to about 0.01 inches. From a start of the plurality of coupling ribs 224 to the second cannula end 212, the cannula body 144 may have a fourteenth length L14 of about 2.4 inches to about 2.8 inches. An external width CW of the cannula tube 214 along a major tube axis 226 between the tapered portion 222 and the plurality of coupling ribs 224 may be about 0.35 inches to about 0.65 inches. With reference to FIG. 17, an external width CW2 of the cannula tube 214 along a minor tube axis 228 between the tapered portion 222 and the plurality of coupling ribs 224 may be about 0.25 inches to about 0.55 inches. Generally, the external width CW2 may be different and less than the external width CW as the cannula tube 214 may have an oval or elliptical shape.

In this regard, with reference to FIG. 19, the cannula tube 214 may have an oval or elliptical cross-sectional shape such that the central lumen 216 that extends through the cannula tube 214 to the second cannula end 212 may be oval or elliptical. The cannula tube 214 may include the major tube axis 226 and the minor tube axis 228. The major tube axis 226 may be perpendicular to the minor tube axis 228. In one example, the major tube axis 226 may have a fifteenth length L15, which may be about 0.3 inches to about 0.5 inches, and the minor tube axis 228 may have a sixteenth length L16, which may be about 0.2 inches to about 0.4 inches. Generally, the fifteenth length L15 and the sixteenth length L16 may be predetermined such that the fifteenth length L15 may be different and greater than the sixteenth length L16 to arrive at the elliptical shape. The elliptical shape of the cannula tube 214, and thus, the central lumen 216 enables the central lumen 216 to be split into two substantially circular or round working lumens when the divider 106 is coupled to the cannula 104. In addition, the elliptical shape of the cannula tube 214 may assist in securing the cannula 104 to the anatomy.

With reference back to FIG. 6, the divider 106 may be insertable into the cannula 104 to divide the central lumen 216 from a single working channel to two substantially discrete or separate working channels. The divider 106 may be composed of a biocompatible material, for example, a biocompatible polymer-based material, including, but not limited to polycarbonate. The divider 106 may be manufactured through any suitable technique, including, but not limited to additive manufacturing. The divider 106 may include a proximal divider end 230, a distal divider end 232 and a divider shaft 234 that connects the proximal divider end 230 to the distal divider end 232.

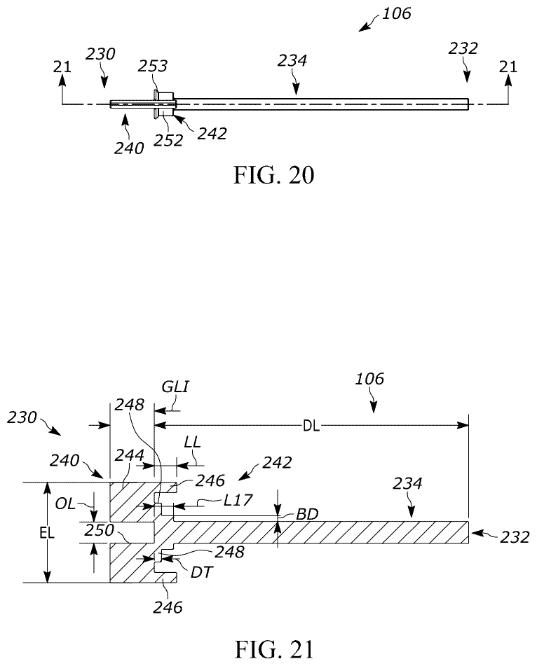

In one example, the proximal divider end 230 may include a pair of insertion tabs 240 and a pair of lumen dividing portions 242. The insertion tabs 240 are on opposite sides of the proximal divider end 230. Each of the insertion tabs 240 may include a tab graspable portion 244, a tab leg 246 and a divider coupling tab 248. The insertion tabs 240 may have a thickness TT of about 0.05 inches to about 0.1 inches (FIG. 23). With reference to FIGS. 20 and 21, the tab graspable portions 244 may define the proximalmost end of the divider 106, and the proximalmost end of the divider 106 may have an end length EL of about 1.0 inches to about 1.3 inches. The tab graspable portions 244 of the insertion tabs 240 may be spaced apart from each other to define an opening 250 that facilitates the insertion of instruments and devices into the cannula body 144. The opening 250 may have an opening length OL of about 0.2 inches to about 0.4 inches. The tab graspable portions 244 may be substantially planar and also provide a visual indicator as to the location of the division of the central lumen 216. The tab graspable portions 244 may extend from the proximalmost end along a graspable length GL1 of about 0.4 inches to 0.6 inches. From the tab graspable portions 244, a remainder of a divider length DL to the distal divider end 232 may be about 3.4 inches to about 3.8 inches.

The tab leg 246 extends axially from the tab graspable portion 244 toward the distal divider end 232. The tab leg 246 may be substantially rectangular (FIG. 6) and may be spaced apart from the divider coupling tab 248 for a distance to enable the tab leg 246 to be positioned along the cap sidewall 154 of the first cap end 150 when the divider 106 is coupled to the cannula 104. This provides additional rigidity to the coupling of the divider 106 to the cannula 104. Generally, the tab leg 246 may extend for a tab leg length LL of about 0.2 inches to 0.3 inches.

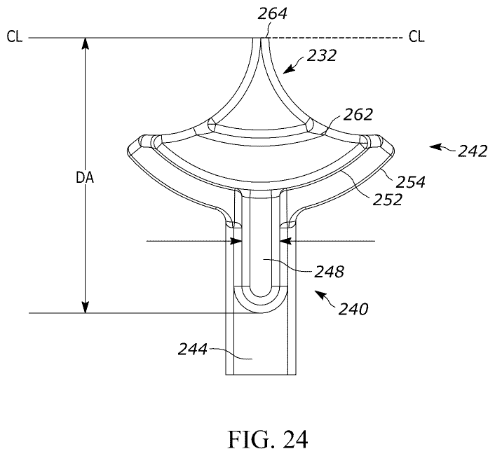

The divider coupling tab 248 may be defined axially from the tab graspable portions 244 and may be adjacent or coupled to the lumen dividing portions 242. The divider coupling tab 248 may have a thickness DT of about 0.05 inches to 0.1 inches, and a width DW of about 0.04 inches to about 0.06 inches (FIG. 24). Generally, the divider coupling tab 248 may be sized and shaped to be received within the coupling grooves 160 of the cannula cap 140. The engagement between the divider coupling tab 248 and the coupling grooves 160 along with the engagement between the tab leg 246 and the cap sidewall 154 of the cannula cap 140 may inhibit the divider 106 from rotating when the divider 106 is coupled to the cannula 104.

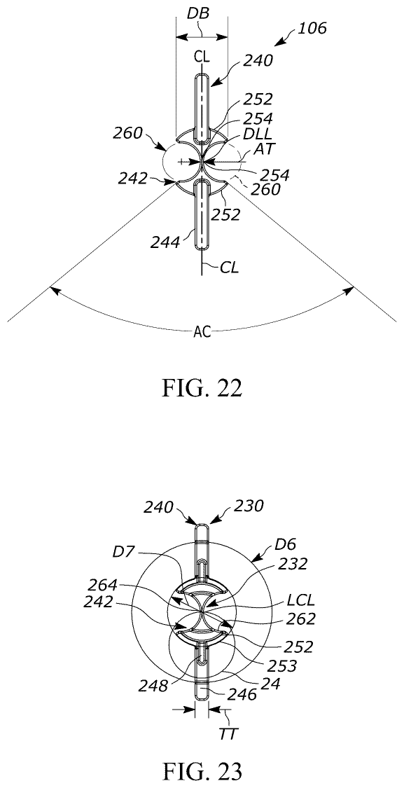

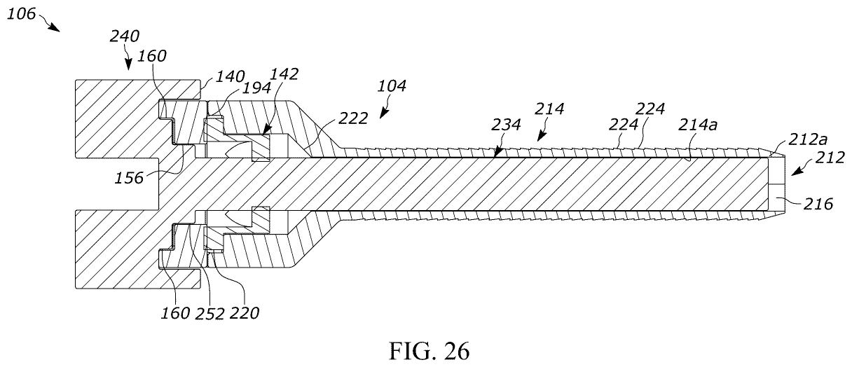

With reference to FIG. 22, a top view of the divider 106 is shown. The lumen dividing portions 242 are substantially triangular in shape, with a base 252 and an apex 254. A perimeter of the base 252 may be curved and may be shaped to be positioned adjacent to or in contact with a portion of the central cap bore 156 (FIG. 26). In one example, the base 252 may have an angle of curvature AC of about 70 degrees to 109 degrees. The base 252 may extend for a distance DB of about 0.25 inches to about 0.45 inches. As best shown in FIG. 21, from the base 252, the lumen dividing portions 242 may extend for a distance BD to assist in coupling the divider 106 within the first cannula end 210. The divider coupling tab 248 and the lumen dividing portions 242 may extend for a length L17, which may be about 0.15 inches to about 0.29 inches. With reference to FIG. 20, the base 252 may also include a chamfer 253 at a proximal end to further assist in positioning the lumen dividing portions 242 within the first cannula end 210.

The apex 254 may be defined at a divider centerline DCL. The lumen dividing portions 242 may be mirror images about the divider centerline DCL such that the apexes 254 meet at the divider centerline DCL. At the divider centerline DCL, the apexes 254 may have a thickness AT of about 0.01 inches to about 0.03 inches. The shape of the lumen dividing portions 242 cooperates with the central cap bore 156 to define a pair of substantially round openings 260 into the cannula tube 214 when the divider 106 is coupled to the cannula 104. In one example, the apexes 254 cooperate to form a portion of a circle having a diameter D5 of about 0.2 inches to about 0.42 inches. It should be noted that while the openings 260 are illustrated as being substantially the same, in other examples, one of the openings 260 may be larger or smaller than the other.

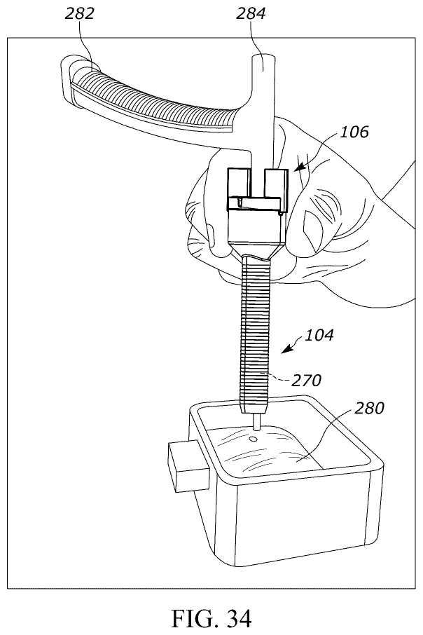

The divider shaft 234 may extend from the lumen dividing portions 242 to the distal divider end 232. The divider shaft 234 may have a substantially similar shape as the lumen dividing portions 242. With reference to FIG. 23, a bottom view of the divider 106 is shown. The divider shaft 234 may include a shaft base 262 and a shaft apex 264. With additional reference to FIG. 24, a detail view taken at 24 on FIG. 23 is shown. The shaft base 262 may be defined radially inward from the base 252 to enable the shaft base 262 to correspond to the shape of the central lumen 216. The divider shaft 234 may define a first channel side 266 opposite a second channel side 268. The shaft apex 264 may be spaced apart from the divider coupling tab 248 by a distance DA, which may be about 0.3 inches to about 0.4 inches. Generally, the first channel side 266 and the second channel side 268 each have a curvature, which may match a curvature of a drill guide 282 (FIG. 34) to stabilize the drill guide 282 during an advancement of a drill 284 along the divider shaft 234.

With reference back to FIG. 23, the shaft apexes 264 may meet at the divider centerline DCL and may have the thickness AT. A circle defined about the divider 106 and bounded by an inner surface of the tab leg 246 may have a diameter D6 of about 0.8 inches to about 1.1 inches. A circle defined about the divider 106 and bounded by an exterior surface of the chamfer 253 may have a diameter D7 of about 0.4 inches to about 0.6 inches.

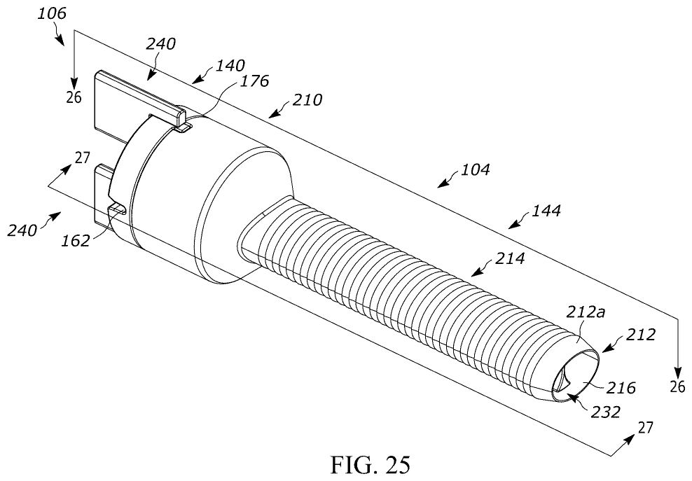

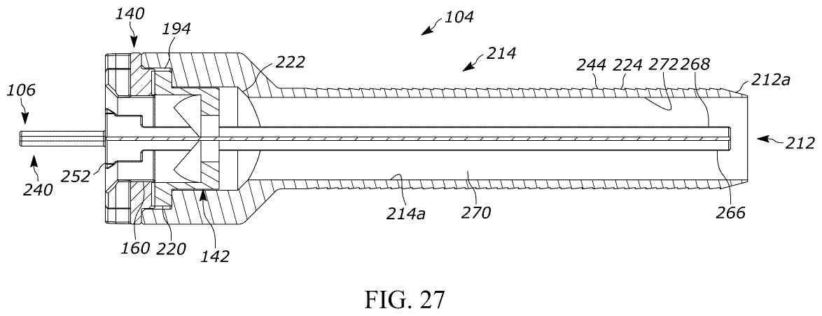

With reference to FIG. 25, the divider 106 is shown coupled to the cannula 104. When coupled to the cannula body 144, the divider shaft 234 may divide or separate the central lumen 216 along a substantial majority of the cannula tube 214. Generally, with reference to FIG. 26, the divider shaft 234 may extend through the cannula tube 214 from proximate the tapered portion 222 to the taper 213 at the second cannula end 212. With reference to FIG. 27, when the divider 106 is coupled to the cannula 104, the divider shaft 234 may divide or separate the central lumen 216 into two independent working channels or a first working channel 270 and a second working channel 272 along a substantial majority of the cannula tube 214. In this example, the first working channel 270 may be bounded by the first channel side 266 of the divider shaft 234 and the interior surface 214a of the cannula tube 214. The second working channel 272 may be bounded by the second channel side 268 of the divider shaft 234 and the interior surface 214a of the cannula tube 214. In one example, the divider 106 may be configured such that when the divider 106 is coupled to the cannula 104, the divider shaft 234 is parallel to or extends along the minor tube axis 228 of the central lumen 216. Generally, the divider shaft 234 may be dimensioned to provide the first working channel 270 and the second working channel 272 with a predetermined shape and volume.

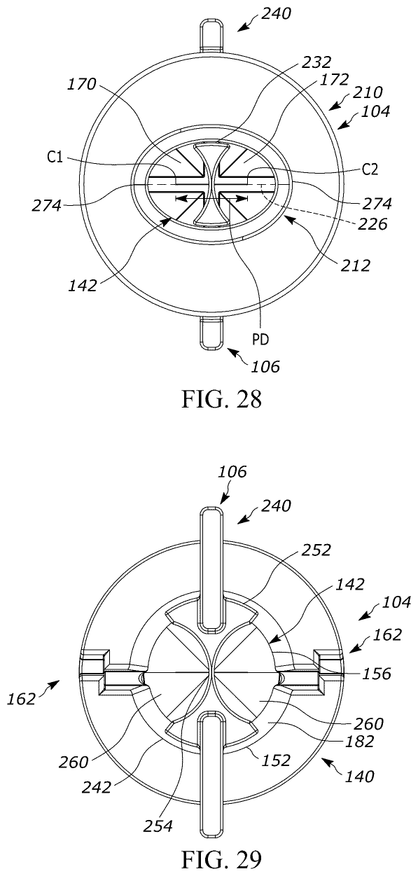

With reference to FIG. 28, an end view of the divider 106 coupled to the cannula 104 is shown. Generally, a center C1 of the first working channel 270 may be spaced a predetermined distance PD from a center C2 of the second working channel 272 so that when medical devices, such as suture anchors, are coupled to the anatomy via the first working channel 270 and the second working channel 272, the medical devices may be spaced apart by the predetermined distance PD. In one example, the predetermined distance PD may be at least 1.0 millimeters. In this example, the divider 106 separates the central lumen 216 into the first working channel 270 and the second working channel 272, each of which may have a substantially circular, round or egg-shaped cross-section. In this example, each of the first working channel 270 and the second working channel 272 may have a substantially equal volume.

Generally, the curvature of the first channel side 266 and the second channel side 268 may be predetermined to match or correspond with an outer curvature of the drill guide 282 as the curvature of the first channel side 266 and the second channel side 268 may assist in orientating the drill guide 282 relative to the respective one of the first working channel 270 and the second working channel 272. The drill guide 282 may follow along the curvature of the first channel side 266 or the second channel side 268 and may not contact the interior surface 214a of the cannula tube 214. The interior surface 214a of the cannula tube 214 may assist in the placement of flexible strands 292 associated with a soft anchor 286 as the respective one of the suture cleats 162 may be aligned with a respective vertex 274 of the major tube axis 226.

Referring to FIG. 29, a top end view of the divider 106 coupled to the cannula 104 is shown. Generally, when the divider 106 is coupled to the cannula 104, the chamfer 253 of the base 252 contacts the chamfer 182 of the central cap bore 156. The lumen dividing portions 242 cooperate with the central cap bore 156 to define the openings 260.

An example method using the cannula system 100 to fix soft tissue to bone to promote healing will be described below. It should be noted that the surgeon may or may not perform the method in the order shown and may perform the method partially or entirely. Moreover, the use of “surgeon” herein is meant to encompass other suitably trained staff that may assist with the method and use of the cannula system 100. The method described below may be performed during a surgical procedure.

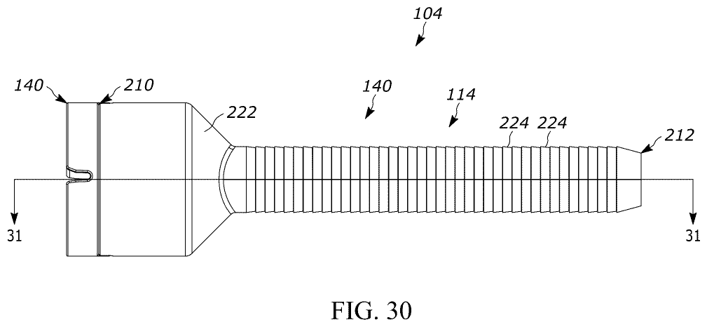

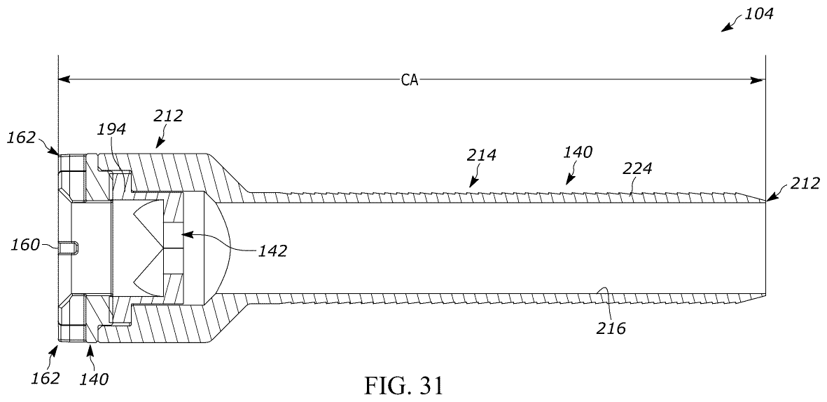

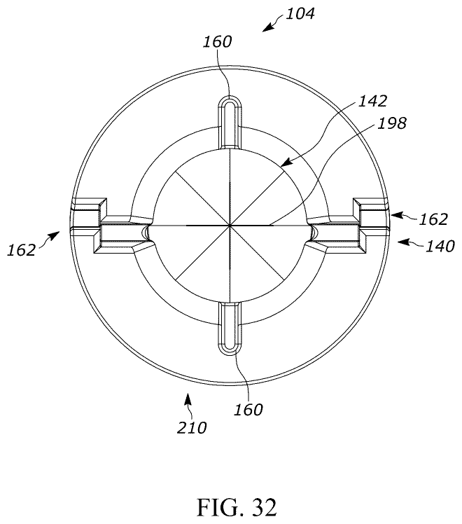

Initially, the cannula 104 may be assembled. With reference to FIGS. 30 and 31, the cannula 104 is shown assembled with the cannula cap 140 and the valve 142 coupled to the cannula body 144. In one example, the valve 142 may be positioned within the central lumen 216 at the first cannula end 210 so that the sealing ring 194 may be received within the receiving channel 220. Generally, with reference to FIG. 32, the cross-slit 198 may be orientated such that the cross-slit 198 may be aligned with the suture cleats 162. With reference back to FIG. 31, the cannula cap 140 may be coupled to the cannula body 144 via any suitable technique, including, but not limited to ultrasonic welding the second cap end 152 to the first cannula end 210 along the coupling surface 180.

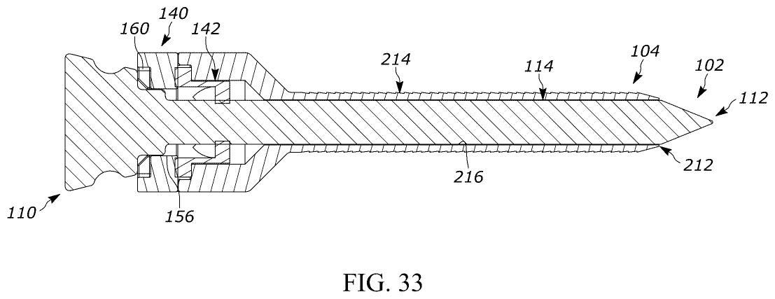

With the cannula 104 assembled, the obturator 102 may be coupled to the cannula 104. For example, with reference to FIG. 33, the obturator 102 may be inserted into the cannula 104 until the collar 134 is received within the central cap bore 156 and the distal obturator end 112 extends beyond the second cannula end 212. With the obturator 102 coupled to the cannula 104, the obturator 102 with the cannula 104 may be inserted percutaneously through the anatomy, proximate the joint to be repaired. In one example, an about 4 millimeters (mm.) to about 5 millimeters (mm.) vertical incision may be made in a skin of the patient, and the obturator 102 with the cannula 104 may be inserted through the incision. In the example of the repair of a Hill-Sach's lesion, the obturator 102 guided by the surgeon to be proximate the humerus. In one example, the obturator 102 may guide the cannula 104 to be above the infraspinatus tendon.

Once the cannula 104 is in the predetermined position in the anatomy, the obturator 102 may be removed, and the cannula 104 may be rotated such that the major tube axis 226 may be perpendicular to the vertical incision. By providing the cannula 104 with the elliptical shape, the orientation of the major tube axis 226 perpendicular to the vertical incision provides additional tension on the skin incision to assist in coupling or retaining the cannula 104 within the anatomy. In addition, the coupling ribs 224 may assist in coupling or retaining the cannula 104 within the anatomy by gripping against the skin and soft tissue.

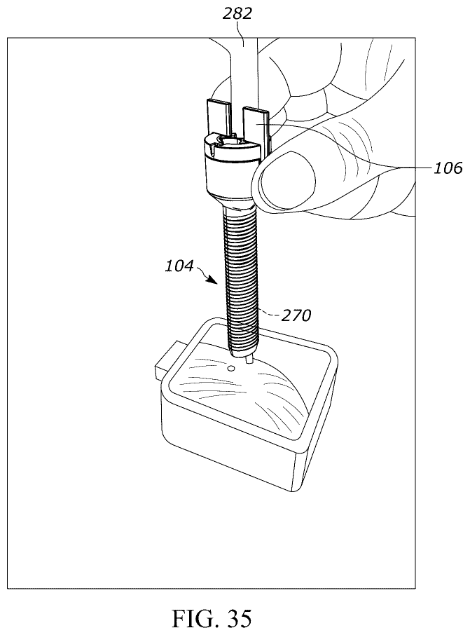

With the cannula 104 positioned at the predetermined location within the anatomy, the divider 106 may be inserted into the cannula 104. With reference to FIGS. 34 and 35, the drill guide 282 may be inserted through one of the first working channel 270 or the second working channel 272 transtendon through the infraspinatus tendon 280. It should be noted that FIGS. 34-44 illustrate the surgical procedure using an artificial infraspinatus tendon 280. In this example, the drill guide 282 may be inserted through the first working channel 270. A drill 284 may be inserted through the drill guide 282 and actuated to drill a first bone socket in the humerus.

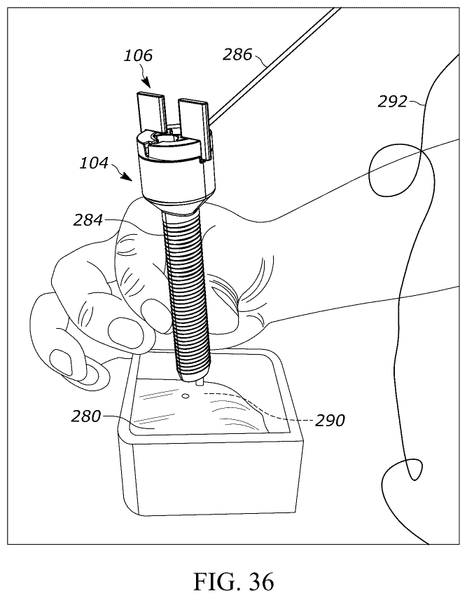

With the first bone socket formed, with reference to FIG. 36, the soft anchor 286, such as a FiberTak® RC Soft Anchor, commercially available from Arthrex, Inc. of Naples, Florida, USA, may be inserted through the one of the first working channel 270 or the second working channel 272 using a suitable soft anchor delivery system, such as that associated with the FiberTak® RC Soft Anchor. In this example, the soft anchor 286 may be inserted through the first working channel 270. Generally, the soft anchor 286 may include a sheath 290 and at least one or the plurality of flexible strands 292. Generally, the sheath 290 may be a tubular sleeve made of a flexible material, such as a braided, woven, or knitted structure made of biocompatible yarns, fibers, filaments, sutures or similar materials, or combinations of these materials. The sheath 290 may be free of barbs or protrusions and may be deformable to secure the soft anchor 286 to the anatomy. The sheath 290 may include a throughbore that receives the flexible strands 292, and includes at least one splice point or opening, which enables the flexible strands 292 to exit and re-enter into the throughbore.

The flexible strands 292 may be coupled to the sheath 290. In one example, the flexible strands 292 may be a suture, including, but not limited to FiberWire®, TigerWire®, or FiberChain® suture commercially available from Arthrex, Inc. of Naples, Florida, USA, although any type of suture may be utilized. The flexible strands 292 may also comprise suture tape, such as FiberTape® commercially available from Arthrex, Inc. of Naples, Florida, USA. Generally, the flexible strands 292 may comprise any soft, biocompatible flexible strands of material. In one example, the flexible strands 292 may include at least a repair suture 294 and a shuttle loop suture 296. The repair suture 294 may be the suture used to couple the soft tissue to the soft anchor 286, and the shuttle loop suture 296 may guide the repair suture 294 into engagement with the sheath 290.

Generally, the flexible strands 292 may be coupled to the sheath 290 such that applying tension to the flexible strands 292 causes the sheath 290 to substantially bunch together into a configuration that secures the sheath 290 in the bone socket. In the bunched together configuration of the sheath 290, the soft anchor 286 may be deployed and resists applied forces so that the surgeon can use the flexible strands 292 to secure the soft tissue to the bone.

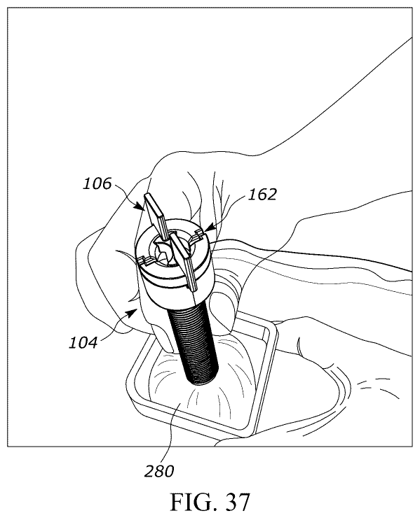

Once the soft anchor 286 is positioned within the first bone socket, the flexible strands 292 may be tensioned into the bunched configuration to set the soft anchor 286 or couple the soft anchor 286 to the first bone socket. With reference to FIG. 37, with the soft anchor 286 set in the first bone socket, all of the flexible strands 292 may be passed through the suture cleat 162 that is in communication with the first working channel 270.

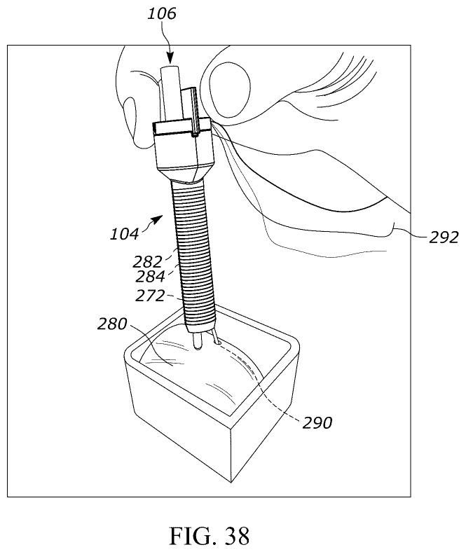

With reference to FIG. 38, the drill guide 282 may be inserted through the other of the first working channel 270 or the second working channel 272 transtendon through the infraspinatus tendon 280. In this example, the drill guide 282 may be inserted through the second working channel 272. The drill 284 may be inserted through the drill guide 282 and actuated to drill a second bone socket in the humerus.

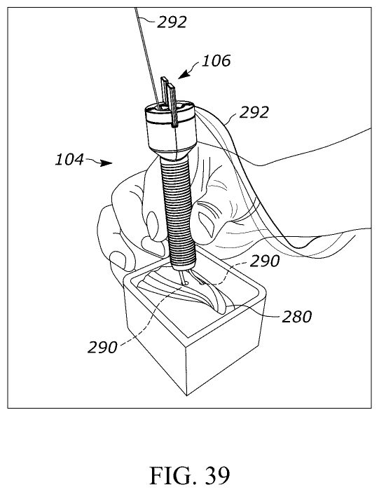

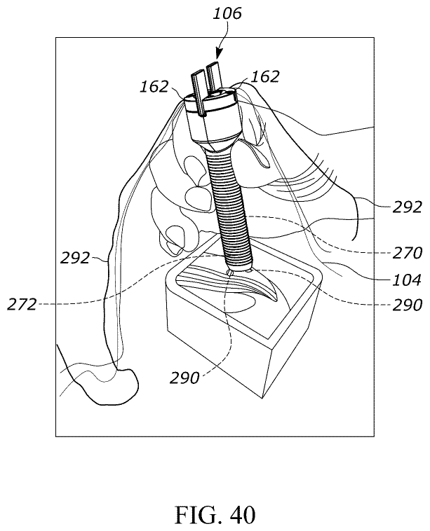

With the second bone socket formed, with reference to FIG. 39, a second one of the soft anchors 286 may be inserted through the other of the first working channel 270 or the second working channel 272. In this example, the second one of the soft anchors 286 may be inserted through the second working channel 272. It should be noted that while the method is described herein as including two of the same soft anchors 286, in other examples, two different anchors may be employed. Once the soft anchor 286 is positioned within the second bone socket, the flexible strands 292 may be tensioned into the bunched configuration to set the soft anchor 286 or couple the soft anchor 286 to the second bone socket. With reference to FIG. 40, with the soft anchor 286 set in the second bone socket, all of the flexible strands 292 may be passed through the suture cleat 162 that is in communication with the second working channel 272.

With the flexible strands 292 of each of the soft anchors 286 coupled to the respective one of the suture cleats 162, the flexible strands 292 may be coupled to the cannula 104 with suitable tension to enable the surgeon to perform other tasks associated with the surgical procedure. For example, the surgeon may complete a Bankart repair while the flexible strands 292 remain cleated to the cannula 104.

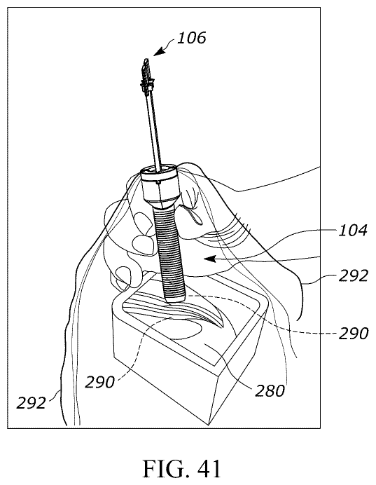

Once the surgeon is ready to tension the soft tissue to the bone, with reference to FIG. 41, the divider 106 may be removed from the cannula 104 before uncleating the flexible strands 292 from the suture cleats 162.

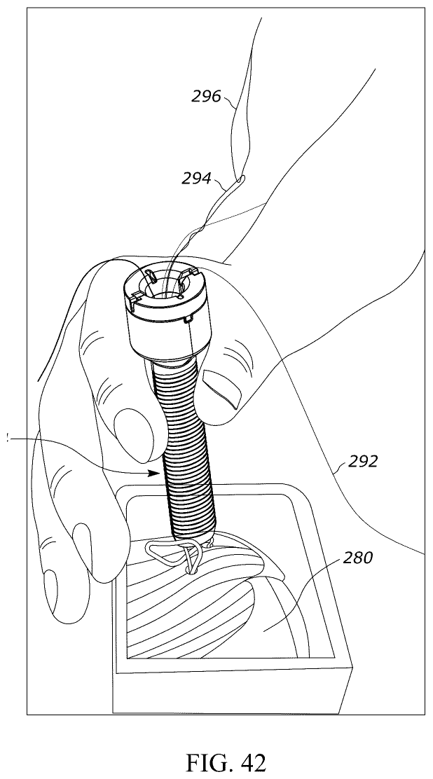

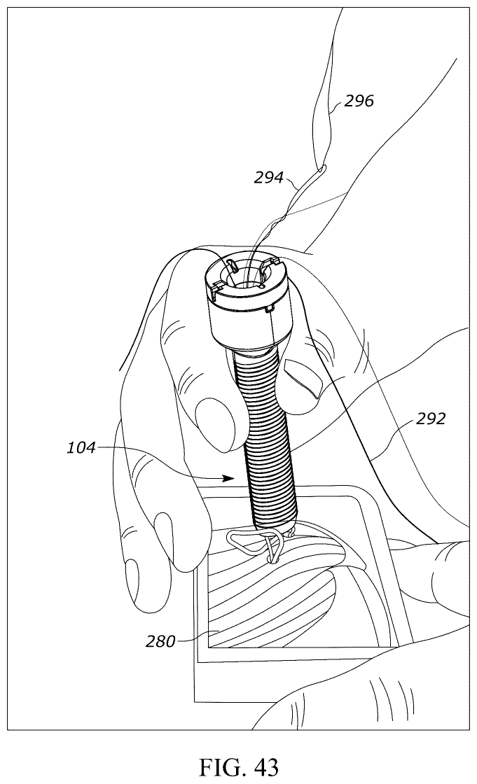

With reference to FIGS. 42 and 43, the repair suture 294 of the soft anchor 286 of the first bone socket may be coupled to or placed into the shuttle loop suture 296 of the soft anchor 286 of the second bone socket. The shuttle loop suture 296 may be pulled to pass the repair suture 294 of the soft anchor 286 of the first bone socket into the sheath 290 of the soft anchor 286 of the second bone socket. This may secure the repair suture 294 of the soft anchor 286 of the first bone socket to the soft anchor 286 of the second bone socket. The repair suture 294 of the soft anchor 286 of the second bone socket may be coupled to or placed into the shuttle loop suture 296 of the soft anchor 286 of the first bone socket. The shuttle loop suture 296 may be pulled to pass the repair suture 294 of the soft anchor 286 of the second bone socket into the sheath 290 of the soft anchor 286 of the first bone socket. This may secure the repair suture 294 of the soft anchor 286 of the second bone socket to the soft anchor 286 of the first bone socket and provides a knotless fixation of the soft tissue to the bone.

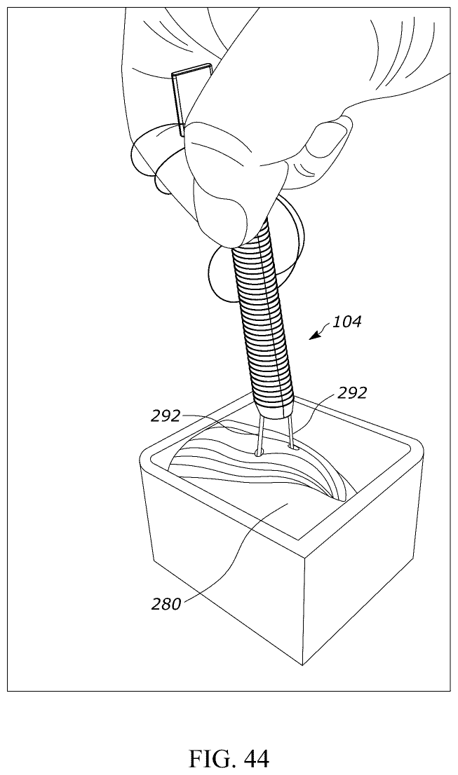

With reference to FIG. 44, with the soft anchors 286 coupled together, the flexible strands 292 and the cannula 104 may be tensioned to apply tension to the soft tissue. Once the soft tissue is tensioned at the predetermined amount, a suture cutter may be used to cut the flexible strands 292. Once the flexible strands 292 are cut to the predetermined length, the cannula 104 may be removed from the vertical incision.

Thus, the cannula system 100 enables a soft tissue repair while protecting the flexible strands 292 used in the soft tissue repair during the surgical procedure. In this regard, by providing the first working channel 270 and the second working channel 272, the drill 284 used to form the second bone socket may be isolated from the soft anchor 286 coupled in the first bone socket. Further, the flexible strands 292 may remain separate from each other until the divider 106 is removed from the cannula 104, which may inhibit the flexible strands 292 from twisting or tangling together inadvertently during the procedure. Moreover, the use of the suture cleats 162 may enable the surgeon to perform other portions of the surgical procedure while the flexible strands 292 remain tensioned and coupled to the cannula 104. The elliptical shape of the cannula tube 214 may provide additional tension on the incision, which may also assist in coupling and retaining the cannula tube 214 within the anatomy during the procedure.

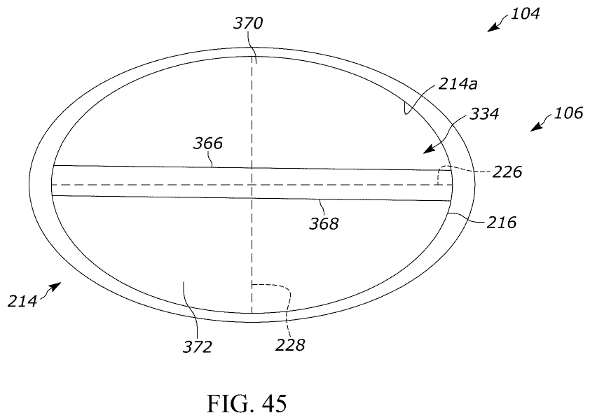

It should be noted that the divider shaft 234 of the divider 106 may be configured differently to separate the central lumen 216 into working channels. For example, with reference to FIG. 45, an end view of a divider shaft 334 is shown coupled to the central lumen 216. In this example, the divider shaft 334 may have a first channel side 366 opposite a second channel side 368. The divider shaft 334 may be configured to be parallel to or extend along the major tube axis 226 of the central lumen 216. The divider shaft 334 may be substantially perpendicular to the minor tube axis 228. When the divider 106 is coupled to the cannula 104, the divider shaft 334 may divide or separate the central lumen 216 into two independent working channels or a first working channel 370 and a second working channel 374 along a substantial majority of the cannula tube 214. The first working channel 370 may be bounded by the first channel side 366 of the divider shaft 334 and the interior surface 214a of the cannula tube 214. The second working channel 372 may be bounded by the second channel side 368 of the divider shaft 334 and the interior surface 214a of the cannula tube 214.

In this example, the first channel side 366 and the second channel side 368 may be substantially flat, planar or devoid of a curvature, such that the first working channel 370 and the second working channel 372 may be substantially semi-elliptical in cross-section. A volume of each of the first working channel 370 and the second working channel 372 may be substantially the same.

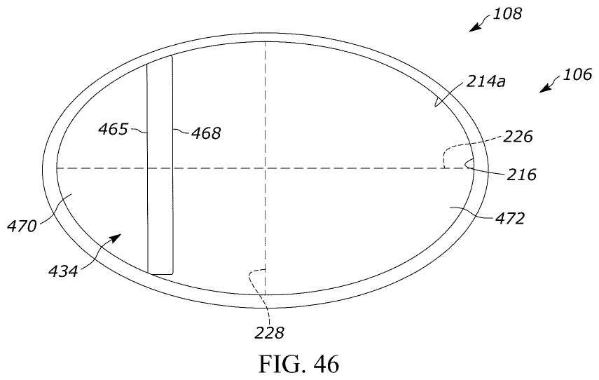

As a further example, with reference to FIG. 46, an end view of a divider shaft 434 is shown coupled to the central lumen 216. In this example, the divider shaft 434 may have a first channel side 466 opposite a second channel side 468. The divider shaft 434 may be configured to be offset from but parallel to the minor tube axis 228 of the central lumen 216. When the divider 106 is coupled to the cannula 104, the divider shaft 434 may divide or separate the central lumen 216 into two independent working channels or a first working channel 470 and a second working channel 472 along a substantial majority of the cannula tube 214. In this example, the first working channel 470 may be bounded by the first channel side 466 of the divider shaft 434 and the interior surface 214a of the cannula tube 214. The second working channel 472 may be bounded by the second channel side 468 of the divider shaft 434 and the interior surface 214a of the cannula tube 214.

In this example, the first channel side 466 and the second channel side 468 may be substantially flat, planar or devoid of a curvature, such that the first working channel 470 and the second working channel 472 may be substantially semi-elliptical in cross-section. A volume of each of the first working channel 470 and the second working channel 472 may be different, and in one example, the volume of the first working channel 470 may be different and less than the volume of the second working channel 472. By providing the first working channel 470 and the second working channel 472 with two different volumes, larger medical instruments or devices, such as graspers, reamers, etc. may be received within the second working channel 472 during a surgical procedure. It should be noted that while first working channel 470 is shown and described herein as having the volume that may be different and less than the second working channel 472, in other examples, the second working channel 472 may have the volume that is different and less than the first working channel 470.

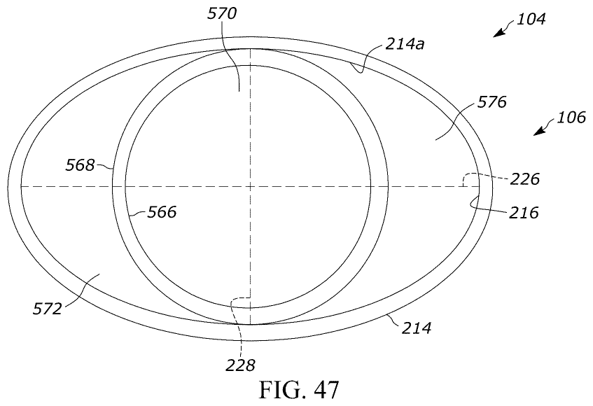

In another example, with reference to FIG. 47, an end view of a divider shaft 534 is shown coupled to the central lumen 216. In this example, the divider shaft 534 may have a first channel side 566 opposite a second channel side 568. The divider shaft 534 may be configured to be parallel to and extend along the minor tube axis 228 of the central lumen 216. When the divider 106 is coupled to the cannula 104, the divider shaft 534 may divide or separate the central lumen 216 into three independent working channels or a first working channel 570, a second working channel 572 and a third working channel 576 along a substantial majority of the cannula tube 214. In this example, the first working channel 570 may be bounded by the first channel side 566 of the divider shaft 534. The second working channel 572 may be bounded by the second channel side 568 of the divider shaft 434 and the interior surface 214a of the cannula tube 214. The third working channel 576 may be bounded by the second channel side 568 of the divider shaft 434 and the interior surface 214a of the cannula tube 214. Generally, the second working channel 572 may be defined opposite the third working channel 576 within the central lumen 216.

In this example, the divider shaft 534 may be annular. The first channel side 566 and the second channel side 568 may be circular, and the first working channel 570 may be circular in cross-section. The second working channel 572 and the third working channel 576 may be substantially semi-elliptical or round in cross-section. A volume of the second working channel 572 and the third working channel 576 may be the same, while a volume of the first working channel 570 may be different and greater than the volume of each of the second working channel 572 and the third working channel 576. By providing the first working channel 570 with a greater volume, larger medical instruments or devices, such as graspers, reamers, etc. may be received within the first working channel 570 during a surgical procedure. In addition, by providing the two smaller second working channel 572 and the third working channel 576, medical devices or implants, such as suture anchors, flexible strands, etc. may be routed through the central lumen 216 and protected from the medical instruments or devices received within the first working channel 570.

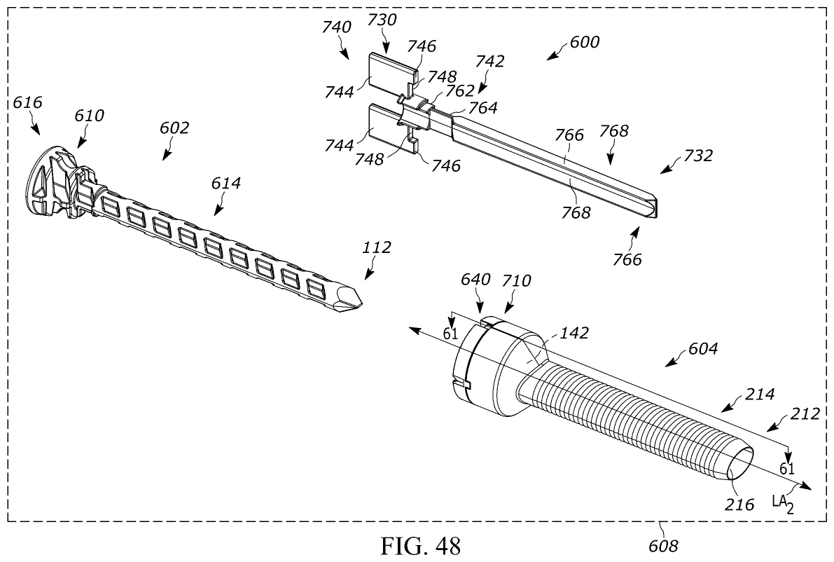

It should be noted in that the cannula system 100 may be configured differently to perform a soft tissue repair surgical procedure, such as a remplissage. With reference to FIG. 48, a cannula system 600 is shown. As the cannula system 600 may include substantially the same or the same features as the cannula system 100 of FIGS. 1-47, the same references numerals will be used to denote these features. In one example, the cannula system 600 may include an obturator 602, a cannula 604 and a divider 606. As will be described, the cannula system 600 enables the percutaneous insertion of the cannula 604 into the anatomy for performing a soft tissue repair surgical procedure, such as a remplissage. Generally, the cannula system 600 may enable a surgeon to perform the soft tissue repair without a line of sight or “blind”, while ensuring that one or more flexible strands or sutures involved in the soft tissue repair are protected during the surgical procedure. The cannula system 600 may also permit the surgeon to perform other portions of the soft tissue repair procedure while retaining tension on the flexible strands or sutures so that the final tensioning of the soft tissue may be completed near the end of the surgical procedure. It should be noted that while the cannula system 600 may be described herein as being using with a remplissage, the cannula system 600 may be used with any suitable surgical procedure, including, but not limited to Hill-Sach's repairs, transtendon repairs (for example, Partial Articular Surface Tendon Avulsion (PASTA) lesion repairs), fixation of soft tissue (ligament, tendon, graft, etc.) to bone, and for any soft tissue repairs including PASTA, labral, rotator cuff, Achilles tendon, biceps and hip repairs, etc. The cannula system 600, the obturator 602, the cannula 604 and the divider 606 may be packaged together in a kit 608, for example.

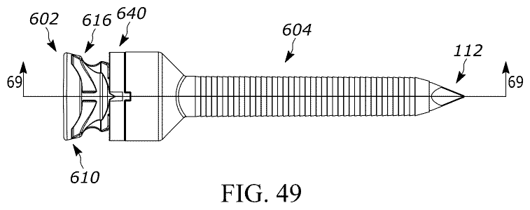

The obturator 602 may assist in the insertion of the cannula 604 into the anatomy. In one example, the obturator 602 may include a proximal obturator end 610, the distal obturator end 112 opposite the proximal obturator end 610, and an obturator shaft 614 that interconnects the proximal obturator end 610 with the distal obturator end 112. The obturator 602 may be composed of a biocompatible material, for example, a biocompatible polymer-based material, including, but not limited to polycarbonate. The obturator 602 may be manufactured through any suitable technique, including, but not limited to additive manufacturing. Generally, the obturator 602 may be clear or transparent, but may comprise a color or color markings. The obturator 602 may be described and illustrated herein as comprising an integral, monolithic or one-piece component, however, in other examples, the proximal obturator end 610 may be separately formed and coupled to the obturator shaft 614 via ultrasonic welding, for example. With reference to FIG. 49, the proximal obturator end 610 extends outwardly from the cannula 604 and may include a graspable portion 616. The graspable portion 616 enables the surgeon to control the obturator 602 and manipulate the obturator 602 to direct the cannula 604 to the predetermined portion of the anatomy.

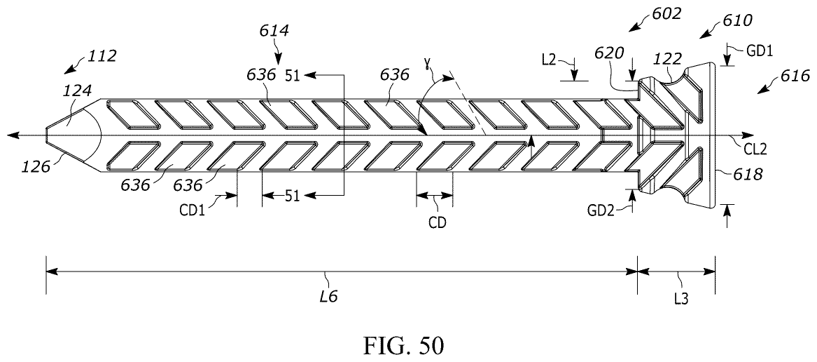

In this example, the graspable portion 616 may be circular and knob-like, however, the graspable portion 616 may have any desired shape to provide a graspable surface or manipulatable portion for use by the surgeon. With reference to FIG. 50, the graspable portion 616 may have the first diameter GD1 at a proximalmost end 618, which may be different and greater than the second diameter GD2 of the graspable portion 616 at an end 620 proximate or at the obturator shaft 614. The fillet 122 may be defined between the proximalmost end 618 and the end 620. The fillet 122 may be spaced radially outward from a centerline CL2 of the obturator 602 by the second length L2. The proximal obturator end 610 extends for the third length L3.

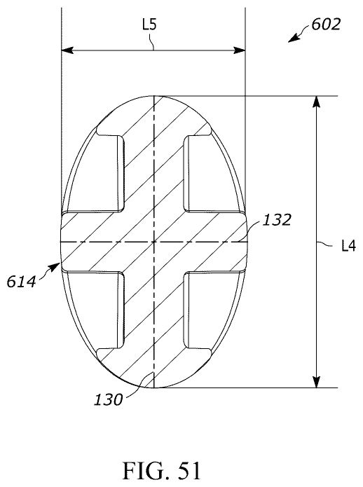

As discussed, the distal obturator end 112 may be tapered to assist in the dilation of tissue as the obturator 602 and the cannula 604 are directed through the anatomy. Generally, with reference to FIG. 51, the obturator shaft 614 of the obturator 602 may be elliptical or oval in cross-section to enable the obturator shaft 614 to be positioned within the cannula 604. The obturator shaft 614 may have the major axis 130 that extends perpendicular to the minor axis 132.

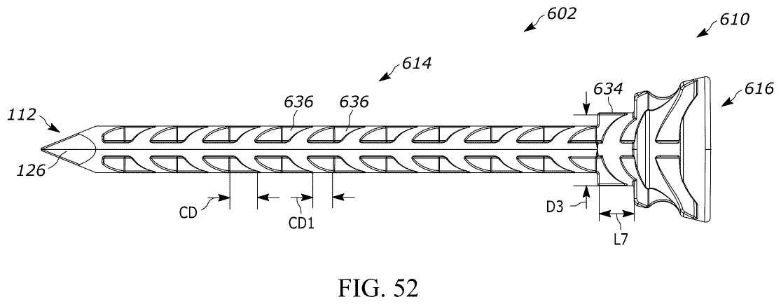

The obturator shaft 614 extends from the distal obturator end 112 to the graspable portion 616 for the sixth length L6. With reference to FIG. 52, the obturator shaft 614 may be coupled to the graspable portion 616 via a collar 634. The collar 634 may have the third diameter D3. The collar 634 may have the seventh length L7. The collar 634 may assist in coupling the cannula 604 to the obturator 602 and may act as a stop that inhibits the advancement of the cannula 604 toward the graspable portion 616 along the obturator shaft 614. The collar 634 may be received within a portion of the cannula 604 to couple the cannula 604 to the obturator 602.

In one example, the obturator 602 may define a plurality of cavities 636 along a length of the obturator 602 from the proximal obturator end 610 to proximate the distal obturator end 112. With reference to FIG. 50, each of the cavities 636 may be defined at an angle γ, and in one example, the angle γ may be about 45 degrees. Each of the cavities 636 may generally extend at the angle γ toward the centerline CL2 for a distance CD of about 0.1 inches (in.) to about 2.0 inches (in.). Generally, each of the cavities 636 may be spaced apart by a distance CD2, which is about 0.05 inches (in.) to about 0.15 inches (in.). In one example, the cavities 636 may be defined along opposed surfaces of the obturator 602, such as a top surface 602a and a bottom surface 602b of the obturator 602. The top surface 602a and the bottom surface 602b may extend parallel to the minor axis 132. Each of the cavities 636 may provide for a reduction in material and a reduction in the mass of the obturator 602.

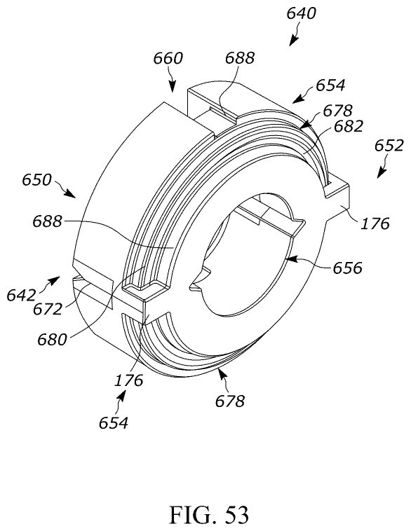

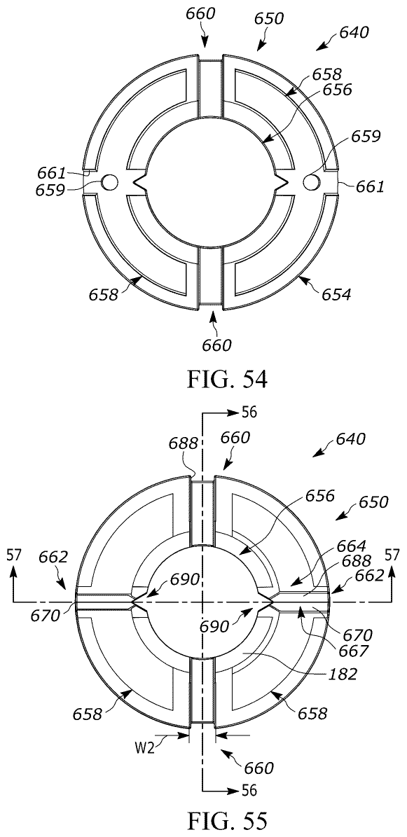

The cannula 604 may include a cap or cannula cap 640, a cap or cannula cap insert 642, the valve 142 and a cannula body 644. With reference to FIG. 53, the cannula cap 640 is shown with the cannula cap insert 642. The cannula cap 640 may be composed of a biocompatible material, for example, a biocompatible polymer-based material, including, but not limited to polycarbonate. The cannula cap 640 may be manufactured through any suitable technique, including, but not limited to additive manufacturing. The cannula cap insert 642 may be composed of a thermoplastic elastomeric material, which may be overmolded onto the cannula cap 640. The cannula cap 640 may have a first cap end 650 opposite a second cap end 652, a cap sidewall 654 that interconnects the first cap end 650 and the second cap end 652, and a central cap bore 656. The cannula cap 640 may define at least one or a pair of mold pockets 658 for forming the cannula cap insert 642 (FIG. 54). The cannula cap 640 may be substantially circular. Generally, the cannula cap 640 may be sized and shaped to be coupled to the cannula body 644 and partially received within the cannula body 644.

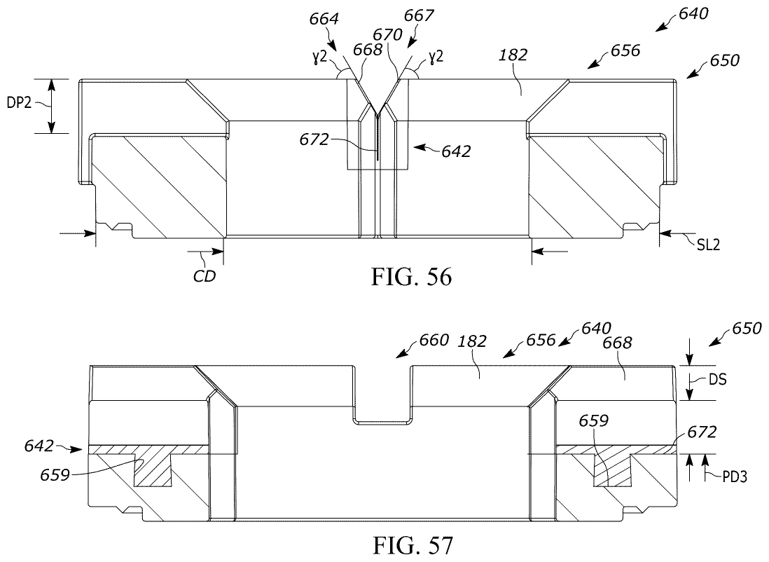

With reference to FIG. 54, a top view of the first cap end 650 is shown without the cannula cap insert 642. The first cap end 650 may have a diameter, which may be about 0.75 inches to about 1.1 inches. The first cap end 650 may include a pair of opposed coupling grooves 660 and the mold pockets 658. In this example, the coupling grooves 660 and the mold pockets 658 may be spaced apart about a perimeter or circumference of the first cap end 650. The coupling grooves 660 may be defined so as to be in communication with the central cap bore 656 and to extend from the central cap bore 656 radially outward to the perimeter of the first cap end 650. The coupling grooves 660 may be sized and shaped to receive a portion of the divider 606 to couple the divider 606 to the cannula 604. Generally, the coupling grooves 660 may be defined to extend along an axis that may be perpendicular to a longitudinal axis LA2 of the cannula 604 (FIG. 48). In one example, the opposed coupling grooves 660 each may have a width W2, which may be about 0.15 inches to about 0.25 inches. With reference to FIG. 56, each of the coupling grooves 660 may be defined to extend for a depth DP2 into the first cap end 650, and in one example, the depth DP2 may be about 0.05 inches to about 0.15 inches. The coupling grooves 660 may also cooperate to define a slot having a slot length SL2 along the first cap end 650 from one coupling groove 660 to the opposite coupling groove 660, which may be about 0.5 inches to about 1.5 inches. The slot may receive the portion of the divider 606 to couple the divider 606 to the cannula 604.

The mold pockets 658 may be opposite each other about the perimeter of first cap end 650. The mold pockets 658 may receive the cannula cap insert 642 to couple the cannula cap insert 642 to the cannula cap 640. In one example, the mold pockets 658 may be substantially semi-circular, and may include a retention bore 659 and a suture slot 661. With reference to FIG. 57, the mold pockets 658 may have a pocket depth PD3, which may be about 0.1 inches to 0.2 inches. The retention bore 659 may be defined through a bottom surface of the cannula cap 640 and may be positioned centrally within the mold pockets 658. In one example, the retention bore 659 may be defined through the suture slot 661 to provided additional strength to the cannula cap insert 642 at the suture slot 661.

With reference to FIG. 54, the suture slots 661 are defined so as to be substantially evenly spaced apart from the coupling grooves 660 about the perimeter of the first cap end 650. In one example, each of the suture slots 661 may have a width, which may be about 0.05 inches to about 0.15 inches. The suture slot 661 may be defined so as to be in communication with the central cap bore 656 and to extend from the central cap bore 656 radially outward through the perimeter of the first cap end 650. Generally, the suture slot 661 may be defined to extend along an axis that may be perpendicular to the longitudinal axis LA2 of the cannula 604 (FIG. 48) and that may be perpendicular to an axis along which the coupling grooves 660 are defined to extend.

With reference to FIG. 55, a top view of the first cap end 650 is shown with the cannula cap insert 642 coupled to the cannula cap 640. Generally, the cannula cap insert 642 may formed within the mold pockets 658 to couple the cannula cap insert 642 to the cannula cap 640. In one example, the cannula cap insert 642 defines a pair of opposed suture retaining slots or suture cleats 662. The suture cleats 662 may be defined in the cannula cap insert 642 by cutting the cannula cap insert 642 with a suitable bladed tool. The suture cleats 662 may receive and retain sutures associated with the surgical procedure. Each of the suture cleats 662 may include a first cleat portion 664 and a second cleat portion 667. The first cleat portion 664 and the second cleat portion 667 may be opposite each other and may extend at outwardly from each other so as to define a substantially V-shape. The first cleat portion 664 and the second cleat portion 667 may be in communication with the central cap bore 656 and may extend radially from the central cap bore 656 to the perimeter of the first cap end 650 along an axis that may be substantially perpendicular to the longitudinal axis LA2 (FIG. 48) of the cannula 604. The axis along which the first cleat portion 664 and the second cleat portion 667 extend to the perimeter may be substantially perpendicular to the axis along which the coupling grooves 660 extend.

In one example, with reference to FIG. 56, the first cleat portion 664 may have a first cleat surface 668, and the second cleat portion 667 may have a second cleat surface 670. A slit 672 may be defined in the cannula cap insert 642 between the first cleat surface 668 and the second cleat surface 670 at a first end to receive and retain a portion of a suture or flexible strand, such as the flexible strands 292. With reference to FIG. 57, the slit 672 may extend for a slit depth DS, which is about 0.14 inches to about 0.20 inches. From the slit 672, the first cleat surface 668 and the second cleat surface 670 may extend outwardly away from each other at an angle γ2 (FIG. 56). With reference back to FIG. 56, the angle γ2 may be defined between the respective one of the first cleat surface 668 and the second cleat surface 670, and a terminal surface of the first cap end 650. The angle γ2 may be about 55 degrees to about 65 degrees.

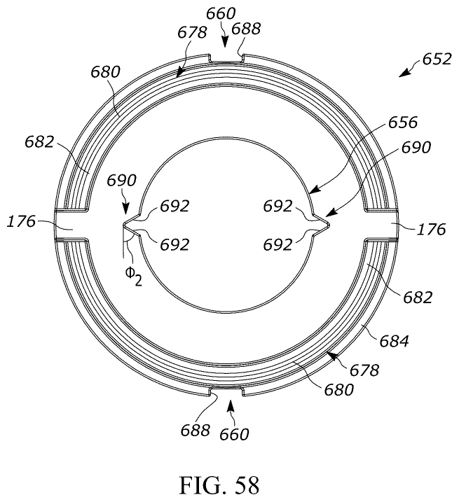

The second cap end 652 may be coupled to the cannula body 644 (FIG. 48). In one example, with reference to FIG. 58, the second cap end 652 includes the pair of coupling tabs 176, which may be aligned with the suture cleats 662 defined on the first cap end 650. The coupling tabs 176 extend substantially radially outward from the cap sidewall 654 at the second cap end 652. The second cap end 652 may also define a pair of coupling ribs 678, which enable the second cap end 652 to be received within and coupled to the cannula body 644. The coupling ribs 678 are recessed relative to the coupling tabs 176 and substantially surround the central cap bore 656 at the second cap end 652. The coupling ribs 678 may extend axially from the second cap end 652. The coupling ribs 678 may be substantially arcuate and may include a lip 680. The lip 680 may assist in coupling the valve 142 to the cannula cap 640. The coupling ribs 678 may be spaced apart from the cap sidewall 654 at the second cap end 652 to define a first receiving recess 682 between the coupling ribs 678 and the cap sidewall 654 at the second cap end 652. The first receiving recess 682 may receive a portion of the valve 142 to couple the valve 142 to the cannula cap 640. A second retaining recess 684 may be defined between the coupling ribs 678 and an outer perimeter of the cannula cap 640 proximate or at the second cap end 652. The second retaining recess 684 may receive a portion of the cannula body 644 to couple the cannula cap 640 to the cannula body 644.

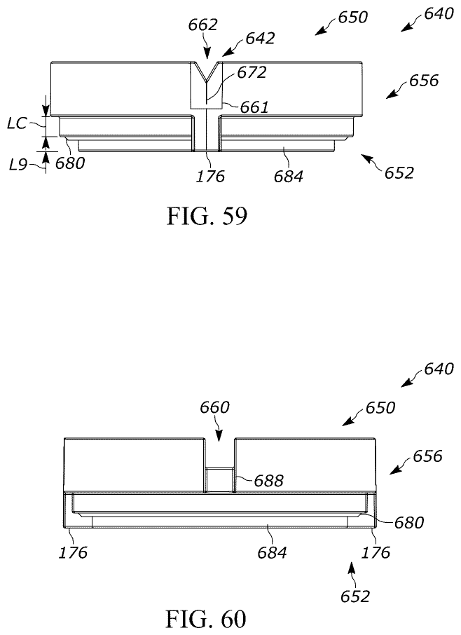

With reference to FIGS. 59 and 60, the cap sidewall 654 interconnects the first cap end 650 with the second cap end 652. The coupling ribs 678 may define a portion of the cap sidewall 654 at the second cap end 652 and may extend for a cap length LC. The cap length LC may be about 0.025 inches to about 0.040 inches. The coupling ribs 678 may be recessed relative to the cap sidewall 654 proximate the first cap end 650 to enable the cannula cap 640 to be received within and coupled to the cannula body 644. In one example, the cannula cap 640 may be coupled to the cannula body 644 via ultrasonic welding. The cap sidewall 654 may also include a recessed portion 686 proximate and at the second cap end 652 to enable the cannula cap 640 to be coupled to the valve 142. The cap sidewall 654 may generally extend for the ninth length L9. With reference to FIG. 60, the cap sidewall 654 may define a pair of notches 688. Each of the notches 688 may correspond with and be in communication with a respective one of the coupling grooves 660. The notches 688 may assist in coupling the divider 606 to the cannula cap 640. The slits 672 may also be defined through the cannula cap insert 642 to extend to the cap sidewall 654 as shown in FIG. 59.

With reference back to FIGS. 56 and 57, the central cap bore 656 may be defined to extend from the first cap end 650 to the second cap end 652. The central cap bore 656 may have the diameter CD. Generally, the central cap bore 656 may be sized to enable instruments associated with the surgical procedure to be inserted through the central cap bore 656 and into the cannula body 644. The central cap bore 656 may also include the chamfer 182 at the first cap end 650 about the central cap bore 656, which may be coupled to and contact the collar 134 of the obturator 602 when the obturator 602 is coupled to the cannula 604. With reference back to FIG. 58, the central cap bore 656 may also include a pair of opposed channels 690, which may be in communication with a respective one of the suture cleats 662. The channels 690 may be defined along the central cap bore 656 from the second cap end 652 to the respective suture cleat 662 and may assist in guiding the flexible strands or sutures into the suture cleats 662. In one example, the opposed channels 690 are each substantially V-shaped and may include a respective pair of channel surfaces 692. Each of the channel surfaces 692 may extend at an angle φ2. In one example, the angle φ2 is defined between a reference line and the respective one of the channel surfaces 692.

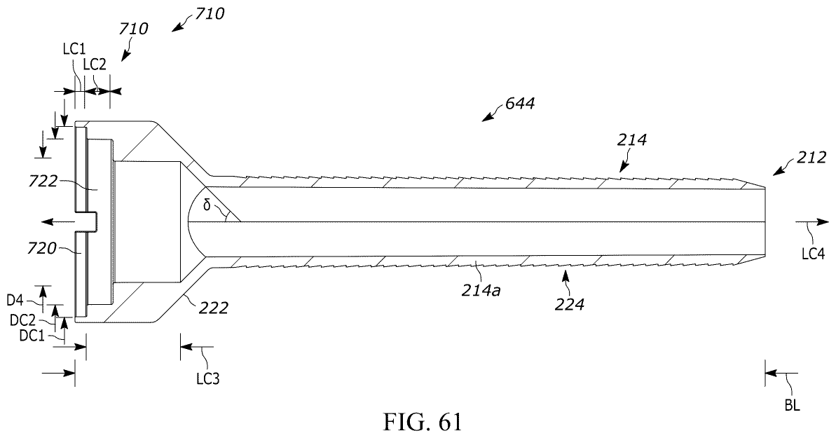

The valve 142 inhibits the flow of fluids out of the cannula body 644 when the cannula body 644 is positioned within the anatomy. With reference back to FIG. 48, the valve 142 may be received within the cannula body 644. The cannula body 644 may be composed of a biocompatible material, for example, a biocompatible polymer-based material, including, but not limited to polycarbonate. The cannula body 644 may be manufactured through any suitable technique, including, but not limited to additive manufacturing. The cannula body 644 may include a first cannula end 710 opposite the second cannula end 212 and the cannula tube 214 that interconnects the first cannula end 710 with the second cannula end 212. The cannula body 644 may also define the central lumen 216, which extends from the first cannula end 710 to the second cannula end 212. The cannula body 644 may have the length BL (FIG. 61).

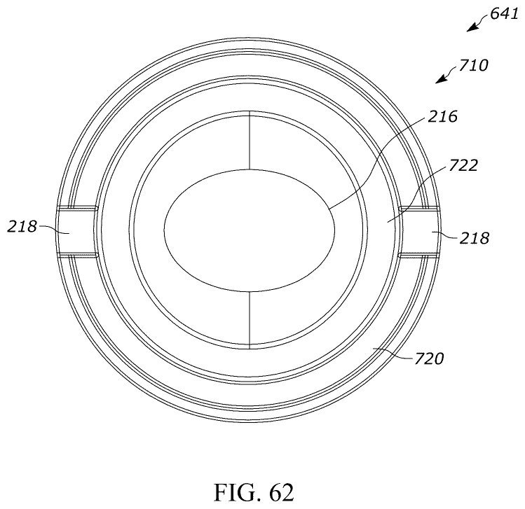

With reference to FIG. 61, the first cannula end 710 may be substantially cup-shaped and may be sized and shaped to be coupled to the cannula cap 640 and the valve 142. With additional reference to FIG. 62, the first cannula end 710 may include the opposed coupling slots 218, a first receiving channel 720, a second receiving channel 722 and the tapered portion 222 (FIG. 61). The coupling slots 218 may receive the coupling tabs 176 of the cannula cap 640.

The first receiving channel 720 may be defined about the central lumen 216 and may be in communication with the opposed coupling slots 218. The first receiving channel 720 may be coupled to or in contact with the coupling ribs 678 of the cannula cap 640 to couple the cannula cap 640 to the cannula body 644. The first receiving channel 720 may have a diameter DC1 of about 0.85 inches to about 1.1 inches and may have a channel length LC1 of about 0.25 inches to 0.35 inches. The second receiving channel 722 may receive the sealing ring 194 of the valve 142 to couple the valve 142 to the cannula body 644. The second receiving channel 722 may have a diameter DC2 of about 0.75 inches to about 0.95 inches and may have a channel length LC2 of about 0.10 inches to 0.20 inches. Between the second receiving channel 722 and the tapered portion 222, the central lumen 216 may have the diameter D4.

The tapered portion 222 may be defined proximate the cannula tube 214 and transitions the first cannula end 710 to the cannula tube 214. The tapered portion 222 may be defined so as to be spaced apart from the first receiving channel 720 by a length LC3, which may be about 0.4 inches to 0.6 inches. In one example, the tapered portion 222 may be defined to have a slope with an angle δ of about 40 degrees to about 50 degrees. The angle δ may be defined between a sidewall of the tapered portion 222 and a centerline LC4 of the cannula body 644.

The second cannula end 212 tapers to form a distalmost end of the cannula 604. The cannula tube 214 may extend from the first cannula end 710 to the second cannula end 212 and may have the interior surface 214a that surrounds a portion of the central lumen 216. The cannula tube 214 may have an oval or elliptical cross-sectional shape such that the central lumen 216 that extends through the cannula tube 214 to the second cannula end 212 may be oval or elliptical. The cannula tube 214 may include the major tube axis 226 and the minor tube axis 228. The elliptical shape of the cannula tube 214, and thus, the central lumen 216 may enable the central lumen 216 to be split into at least two substantially circular or round working lumens when the divider 606 is coupled to the cannula 604. In addition, the elliptical shape of the cannula tube 214 may assist in securing the cannula 604 to the anatomy.

With reference back to FIG. 48, the divider 606 may be insertable into the cannula 604 to divide the central lumen 216 from a single working channel to at least two substantially discrete or separate working channels. The divider 606 may be composed of a biocompatible material, for example, a biocompatible polymer-based material, including, but not limited to polycarbonate. The divider 606 may be manufactured through any suitable technique, including, but not limited to additive manufacturing. The divider 606 may include a proximal divider end 730, a distal divider end 732 and a divider shaft 734 that connects the proximal divider end 730 to the distal divider end 732.

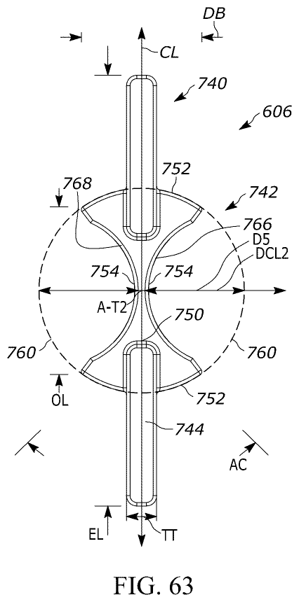

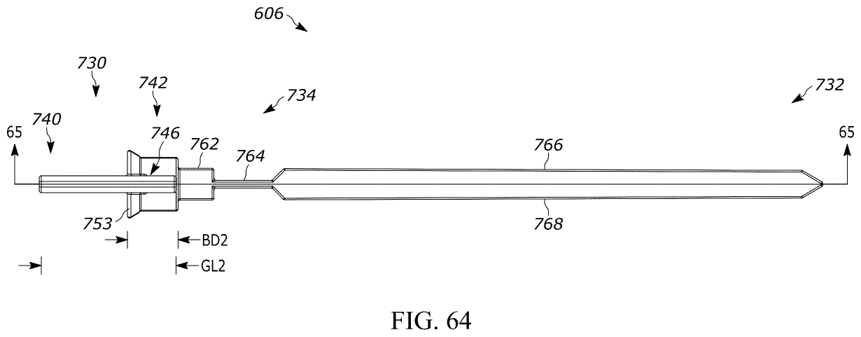

In one example, the proximal divider end 730 may include a pair of insertion tabs 740 and a pair of lumen dividing portions 742. The insertion tabs 740 are on opposite sides of the proximal divider end 730. Each of the insertion tabs 740 may include a tab graspable portion 744, a tab leg 746 and a divider coupling recess 748. With reference to FIG. 63, the insertion tabs 740 may have the thickness TT. The tab graspable portions 744 may define the proximalmost end of the divider 606, and the proximalmost end of the divider 606 may have the end length EL. The tab graspable portions 744 of the insertion tabs 740 may be spaced apart from each other to define an opening 750 that facilitates the insertion of instruments and devices into the cannula body 644. The opening 750 may have the opening length OL. The tab graspable portions 744 may be substantially planar and also provide a visual indicator as to the location of the division of the central lumen 216. With reference to FIG. 64, the tab graspable portions 744 may extend from the proximalmost end along a graspable length GL2 of about 0.5 inches to 0.7 inches.

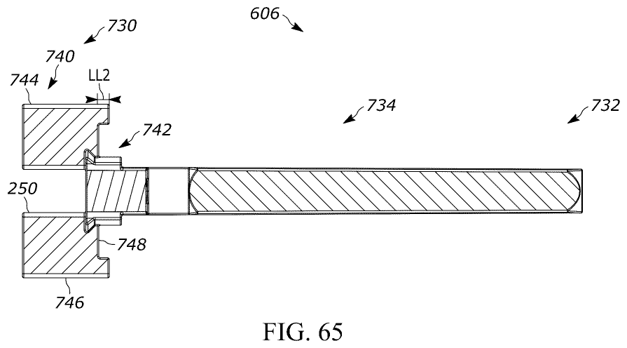

The tab leg 746 extends axially from the tab graspable portion 744 toward the distal divider end 732. With reference to FIG. 65, the tab leg 746 may be substantially rectangular (FIG. 59) and may be spaced apart from the portions 742 by the divider coupling recess 748 to enable the tab leg 746 to be positioned along the cap sidewall 654 of the first cap end 650 when the divider 606 is coupled to the cannula 604. This provides additional rigidity to the coupling of the divider 606 to the cannula 604. Generally, the tab leg 746 may extend for a tab leg length LL2 of about 0.05 inches to 0.2 inches. Each of the tab legs 746 may be received within a respective one of the notches 688 of the cannula cap 640 when the divider 606 is coupled to the cannula 604.

The divider coupling recess 748 may be defined adjacent or coupled to the lumen dividing portions 742. Generally, the divider coupling recess 748 may be sized and shaped to be received within the coupling grooves 660 of the cannula cap 640. The engagement between the divider coupling recess 748 and the coupling grooves 660 along with the engagement between the tab leg 746 and the notches 688 of the cannula cap 640 may inhibit the divider 606 from rotating when the divider 606 is coupled to the cannula 604.

With reference to FIG. 63, a top view of the divider 606 is shown. The lumen dividing portions 742 are substantially triangular in shape, with a base 752 and an apex 754. A perimeter of the base 752 may be curved and may be shaped to be positioned adjacent to or in contact with a portion of the central cap bore 656 (FIG. 55). In one example, the base 752 may have the angle of curvature AC. The base 752 may extend for the distance DB. As best shown in FIG. 64, from the base 752, the lumen dividing portions 742 may extend for a distance BD2 to assist in coupling the divider 606 within the first cannula end 710. The base 752 may also include a chamfer 753 at a proximal end to further assist in positioning the lumen dividing portions 742 within the first cannula end 710.

With reference back to FIG. 63, the apex 754 may be defined at a divider centerline DCL2. The lumen dividing portions 742 may be mirror images about the divider centerline DCL2 such that the apexes 754 meet at the divider centerline DCL2. At the divider centerline DCL2, the apexes 754 may have a thickness AT2 of about 0.03 inches to about 0.05 inches. The shape of the lumen dividing portions 742 cooperates with the central cap bore 656 to define a pair of substantially round openings 760 into the cannula tube 214 when the divider 606 is coupled to the cannula 604. In one example, the apexes 754 cooperate to form a portion of a circle having the diameter D5. It should be noted that while the openings 760 are illustrated as being substantially the same, in other examples, one of the openings 760 may be larger or smaller than the other.

With reference back to FIG. 48, the divider shaft 734 may extend from the lumen dividing portions 742 to the distal divider end 732. The divider shaft 734 may include a shaft connector 762 and a shaft neck 764. With reference to FIG. 64, the shaft connector 762 may be defined radially inward from the base 752 to correspond to the shape of the central lumen 216. The shaft connector 762 and the portions 742 cooperate with the central lumen 216 to couple the divider 606 to the cannula 604.

With reference to FIG. 64, the shaft neck 764 may be defined between the shaft connector 762 and a pair of channels 765 defined by the divider shaft 734. In one example, the shaft neck 764 may be substantially rectangular and planar, which may cooperate with the valve 142 to maintain the valve opening 196 closed when the divider 606 is coupled to the cannula 604. Stated another way, the shaft neck 764 may cooperate with leaves 200 of the valve 142 to close the valve opening 196 when the divider 606 is coupled to the cannula 604. Generally, the channels 765 may include a first channel side 766 opposite a second channel side 768. In one example, the first channel side 766 and the second channel side 768 each have a curvature, which may match a curvature of the drill guide 282 (FIG. 34) to stabilize the drill guide 282 during an advancement of a drill 284 along the divider shaft 734.

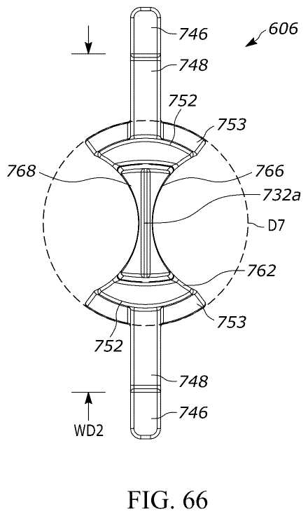

With reference to FIG. 66, a bottom view of the divider 606 is shown. The divider 606 may have a width WD2 that is bounded by an inner surface of each of the tab legs 746, which may be about 0.65 inches to about 1.05 inches. A circle defined about the divider 606 and bounded by an exterior surface of the chamfer 753 may have the diameter D7. In one example, the distal divider end 732 may terminate in a pointed tip 732a. The pointed tip 732a may facilitate the insertion of the divider 606 into the cannula 604 during use.

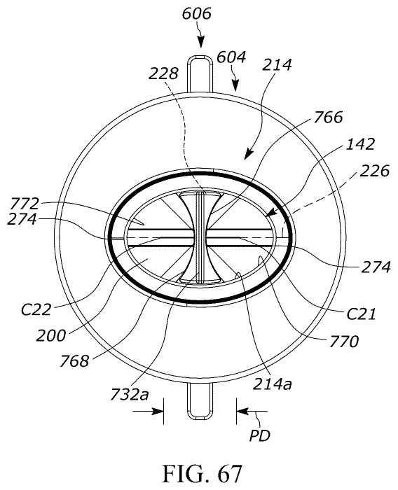

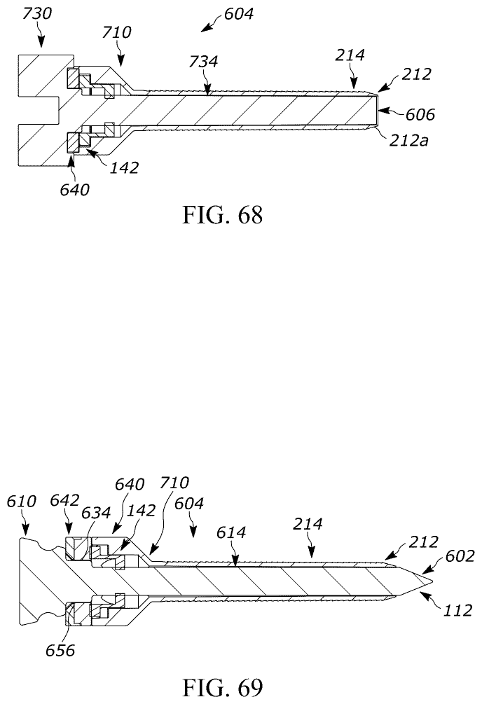

When coupled to the cannula body 644, the divider shaft 734 may divide or separate the central lumen 216 along a substantial majority of the cannula tube 214, as shown in FIGS. 67 and 68. Generally, the divider shaft 734 may extend through the cannula tube 214 from proximate the tapered portion 222 to the taper 212a at the second cannula end 212 (FIG. 68). When the divider 606 is coupled to the cannula 604, the divider shaft 734 may divide or separate the central lumen 216 into two independent working channels or a first working channel 770 and a second working channel 772 along a substantial majority of the cannula tube 214 (FIG. 67). In this example, with reference to FIG. 67, the first working channel 770 may be bounded by the first channel side 766 of the divider shaft 734 and the interior surface 214a of the cannula tube 214. The second working channel 772 may be bounded by the second channel side 768 of the divider shaft 734 and the interior surface 214a of the cannula tube 214. In one example, the divider 606 may be configured such that when the divider 606 is coupled to the cannula 604, the divider shaft 734 may be parallel to or extends along the minor tube axis 228 of the central lumen 216. Generally, the divider shaft 734 may be dimensioned to provide the first working channel 770 and the second working channel 772 with a predetermined shape and volume.

Generally, a center C21 of the first working channel 770 may be spaced the predetermined distance PD from a center C22 of the second working channel 772 so that when medical devices, such as suture anchors, are coupled to the anatomy via the first working channel 770 and the second working channel 772, the medical devices may be spaced apart by the predetermined distance PD. In this example, the divider 606 separates the central lumen 216 into the first working channel 770 and the second working channel 772, each of which may have a substantially circular, round or egg-shaped cross-section. In this example, each of the first working channel 770 and the second working channel 772 may have a substantially equal volume.

Generally, the curvature of the first channel side 766 and the second channel side 768 may be predetermined to match or correspond with an outer curvature of the drill guide 282 as the curvature of the first channel side 766 and the second channel side 768 may assist in orientating the drill guide 282 relative to the respective one of the first working channel 770 and the second working channel 772. The drill guide 282 may follow along the curvature of the first channel side 766 or the second channel side 768 and may not contact the interior surface 214a of the cannula tube 214. The interior surface 214a of the cannula tube 214 may assist in the placement of flexible strands 292 associated with the soft anchor 286 as the respective one of the suture cleats 662 may be aligned with a respective vertex 274 of the major tube axis 226. When the divider 606 is coupled to the cannula 604, the chamfer 753 of the base 752 may contact the chamfer 182 of the central cap bore 656. The lumen dividing portions 742 cooperate with the central cap bore 656 to define the openings 760 (FIG. 63).

An example method using the cannula system 600 to fix soft tissue to bone to promote healing will be described below. It should be noted that the surgeon may or may not perform the method in the order shown and may perform the method partially or entirely. The method described below may be performed during a surgical procedure.

Initially, with reference to FIG. 48, the cannula 604 may be assembled with the cannula cap 640 and the valve 142 coupled to the cannula body 644. The cannula cap insert 642 may be overmolded onto the cannula cap 640 and the suture cleats 662 may be defined in the cannula cap insert 642. The valve 142 may be positioned within the central lumen 216 at the first cannula end 710 so that the sealing ring 194 may be received within the second receiving channel 722. Generally, the cross-slit 198 may be orientated such that the cross-slit 198 may be aligned with the suture cleats 662. The cannula cap 640 may be coupled to the cannula body 644 via any suitable technique, including, but not limited to ultrasonic welding the second cap end 652 to the first cannula end 710 along the coupling ribs 678. With the cannula 604 assembled, the obturator 602 may be coupled to the cannula 604. For example, with reference to FIG. 69, the obturator 602 may be inserted into the cannula 604 until the collar 634 may be received within the central cap bore 656 and the distal obturator end 112 may extend beyond the second cannula end 212.

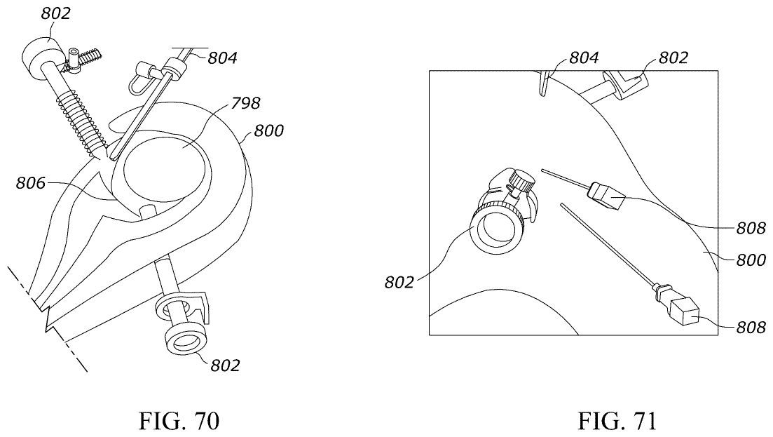

With reference to FIG. 70, an anatomy 800 may be prepared for the surgical procedure. In the following examples, the cannula system 600 may be used to perform a remplissage procedure to repair a Hill-Sach's defect on a humeral head 798, however, as discussed, the cannula system 600 may be used with any suitable surgical procedure. In one example, the anatomy 800 may be prepared to include two additional working cannulas 802. The working cannulas 802 may include, but are not limited to, one or more of a Twist-In™ Cannula, Gemini Cannula, or the like, which may be commercially available from Arthrex, Inc. of Naples, Florida, United States of America. A viewing device 804 may also be coupled to the anatomy to provide a portal for the surgeon to view the surgical procedure. The viewing device 804 may include, but is not limited to, an arthroscope, a NanoScope™ commercially available from Arthrex, Inc. of Naples, Florida, United States of America or the like. In one example, the viewing device 804 may be positioned to provide an anterior superior viewing portal in an area between the humeral head 798 and a glenoid 806 as shown. In one example, the viewing device 804 may include a 70-degree scope lens. The surgeon may use the viewing device 804 to locate the Hill-Sachs lesion and prepare a bone defect to create a bleeding bed to enhance tissue healing to bone.

With reference to FIG. 71, one or more distension needles 808 may be inserted into the anatomy 800. In one example, a pair of the distension needles 808 may be used to locate a superior end and an inferior end of the Hill-Sachs defect. In this example, the cannula 604 may comprise a posterior lateral cannula, which may be inserted at a mid-point between the two one or more distension needles 808. After the position of the cannula 604 is determined, an about 4 millimeters (mm.) to about 5 millimeters (mm.) vertical incision may be made in a skin of the patient, and the obturator 602 with the cannula 604 may be inserted through the incision. In one example, the obturator 602 may guide the cannula 604 to be above the infraspinatus tendon 280.

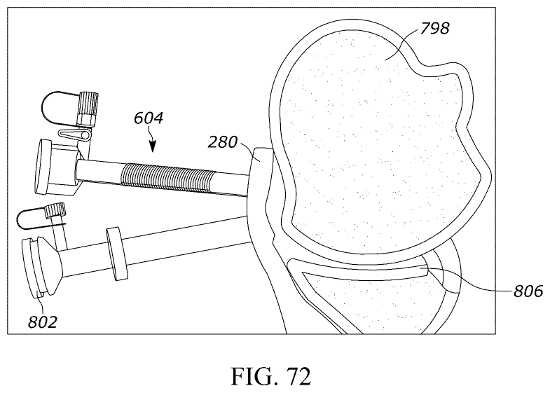

Once the cannula 604 is in the predetermined position in the anatomy, the obturator 602 may be removed, and the cannula 604 may be rotated such that the major tube axis 226 may be perpendicular to the vertical incision. By providing the cannula 604 with the elliptical shape, the orientation of the major tube axis 226 perpendicular to the vertical incision provides additional tension on the skin incision to assist in coupling or retaining the cannula 604 within the anatomy. In addition, the coupling ribs 224 may assist in coupling or retaining the cannula 604 within the anatomy by gripping against the skin and soft tissue. Generally, with reference to FIG. 72, the cannula 604 may be positioned between a deltoid and the infraspinatus tendon 280. The surgeon may sweep the cannula 604 side to side against the infraspinatus tendon 280 to detach bursal tissue creating space between the deltoid and the infraspinatus tendon 280.

With the cannula 604 positioned at the predetermined location within the anatomy, the divider 606 may be inserted into the cannula 604. It should be noted that the divider 606 may also be inserted after the insertion of a first one of the soft anchors 286. If the divider 606 is inserted after the insertion of the first one of the soft anchors 286, the flexible strands 292 associated with the soft anchor 286 may be coupled to the suture cleats 662 prior to inserting the divider 606.

The drill guide 282 (FIG. 35) may be inserted through one of the first working channel 770 or the second working channel 772 (or through the central lumen 216 if the divider 606 is not coupled to the cannula 604) transtendon through the infraspinatus tendon 280. In this example, the drill guide 282 may be inserted through the first working channel 770. Generally, the drill guide 282 may be positioned through the first working channel 770 such that a tip of the drill guide 282 may be at an inferior position on the Hill-Sach's defect. In certain instances, a cannulated needle and/or a k-wire may be used to localized and dilate the insertion position through the infraspinatus tendon 280. The drill 284 may be inserted through the drill guide 282 and actuated to drill a first bone socket in the humerus.

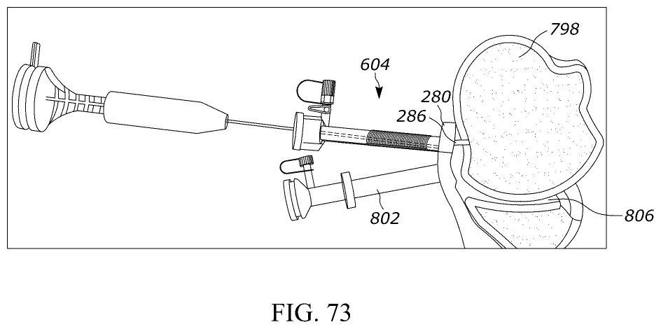

With the first bone socket formed, with reference to FIG. 73, the soft anchor 286 may be inserted through the one of the first working channel 770 or the second working channel 772. In this example, the soft anchor 286 may be inserted through the first working channel 770. Once the soft anchor 286 is positioned within the first bone socket, the flexible strands 292 may be tensioned into the bunched configuration to set the soft anchor 286 or couple the soft anchor 286 to the first bone socket. With reference to FIG. 37, with the soft anchor 286 set in the first bone socket, all of the flexible strands 292 may be passed through and coupled to the suture cleat 662 that is in communication with the first working channel 770. If the divider 606 was not coupled to the cannula 604 for the insertion of the soft anchor 286, all of the flexible strands 292 may be passed through and coupled to the suture cleat 662 that is aligned with the inserted soft anchor 286.

If the divider 606 was not used for the insertion of the first soft anchor 286, the divider 606 may be coupled to the cannula 604. The drill guide 282 may be inserted through the other of the first working channel 770 or the second working channel 772 transtendon through the infraspinatus tendon 280. In this example, the drill guide 282 may be inserted through the second working channel 772. Generally, the drill guide 282 may be positioned through the second working channel 772 such that a tip of the drill guide 282 may be at a superior position on the Hill-Sach's defect. In certain instances, a cannulated needle and/or a k-wire may be used to localized and dilate the insertion position through the infraspinatus tendon 280. The drill 284 may be inserted through the drill guide 282 and actuated to drill a second bone socket in the humerus.

With the second bone socket formed, a second one of the soft anchors 286 may be inserted through the other of the first working channel 770 or the second working channel 772. In this example, the second one of the soft anchors 286 may be inserted through the second working channel 772. It should be noted that while the method is described herein as including two of the same soft anchors 286, in other examples, two different anchors may be employed. Once the soft anchor 286 is positioned within the second bone socket, the flexible strands 292 may be tensioned into the bunched configuration to set the soft anchor 286 or couple the soft anchor 286 to the second bone socket. With the soft anchor 286 set in the second bone socket, all of the flexible strands 292 may be passed through the suture cleat 662 that is in communication with the second working channel 772. The soft anchor 286 of the first bone socket may be an inferior anchor 286a, and the soft anchor 286 of the second bone socket may be a superior anchor 286b.

With the flexible strands 292 of each of the soft anchors 286 coupled to the respective one of the suture cleats 662, the flexible strands 292 may be coupled to the cannula 604 with suitable tension to enable the surgeon to perform other tasks associated with the surgical procedure. For example, the surgeon may complete a Bankart repair while the flexible strands 292 remain cleated to the cannula 604.

Once the surgeon is ready to tension the soft tissue to the bone, the divider 606 may be removed from the cannula 604 before uncleating the flexible strands 292 from the suture cleats 162.

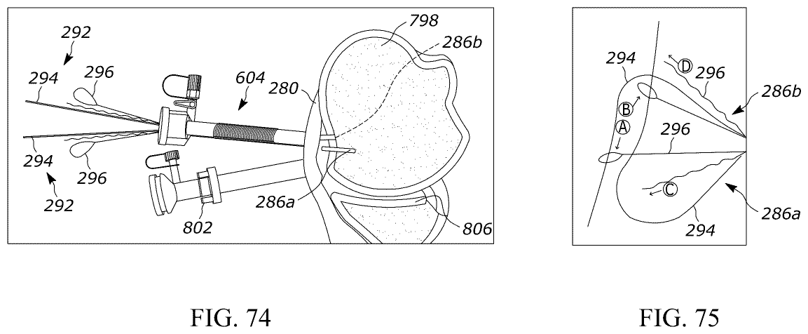

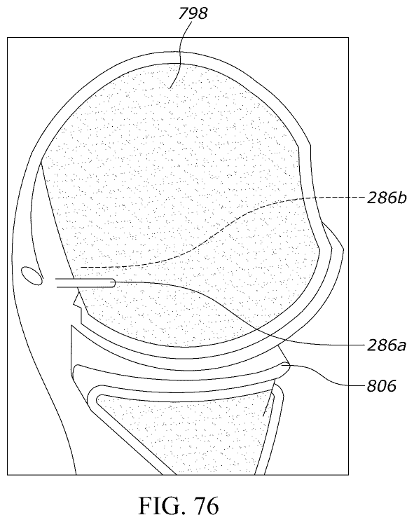

With reference to FIGS. 74 and 75, the repair suture 294 of the soft anchor 286 of the first bone socket may be coupled to or placed into the shuttle loop suture 296 of the soft anchor 286 of the second bone socket (A on FIG. 75). An opposite end of the shuttle loop suture 296 of the soft anchor 286 of the second bone socket may be pulled to pass the repair suture 294 of the soft anchor 286 of the first bone socket into the sheath 290 of the soft anchor 286 of the second bone socket (B on FIG. 75). This may secure the repair suture 294 of the soft anchor 286 of the first bone socket to the soft anchor 286 of the second bone socket. The repair suture 294 of the soft anchor 286 of the second bone socket may be coupled to or placed into the shuttle loop suture 296 of the soft anchor 286 of the first bone socket (C on FIG. 75). An opposite end of the shuttle loop suture 296 of the soft anchor 286 of the first bone socket may be pulled to pass the repair suture 294 of the soft anchor 286 of the second bone socket into the sheath 290 of the soft anchor 286 of the first bone socket (D on FIG. 75). This may secure the repair suture 294 of the soft anchor 286 of the second bone socket to the soft anchor 286 of the first bone socket.

With the soft anchors 286 coupled together, the flexible strands 292 and the cannula 604 may be tensioned to apply tension to the soft tissue. Once the soft tissue is tensioned at the predetermined amount, a suture cutter may be used to cut the flexible strands 292. Once the flexible strands 292 are cut to the predetermined length, the cannula 604 may be removed from the vertical incision. The cannula system 600 may provide a knotless fixation of the soft tissue to the bone as shown in FIG. 76.

Thus, the cannula system 100, 600 enables a soft tissue repair while protecting the flexible strands 292 used in the soft tissue repair during the surgical procedure. In this regard, by providing at least the first working channel 270, 770 and the second working channel 272, 772, the drill 284 used to form the second bone socket may be isolated from the soft anchor 286 coupled in the first bone socket. Further, the flexible strands 292 may remain separate from each other until the divider 106, 606 is removed from the cannula 104, 604, which may inhibit the flexible strands 292 from twisting or tangling together inadvertently during the procedure. Moreover, the use of the suture cleats 162, 662 may enable the surgeon to perform other portions of the surgical procedure while the flexible strands 292 remain tensioned and coupled to the cannula 104, 604. The elliptical shape of the cannula tube 214 may provide additional tension on the incision, which may also assist in coupling and retaining the cannula tube 214 within the anatomy during the procedure.

It should be noted that in certain examples, the divider 106, 606 may be provided with the divider shaft 334, the divider shaft 434 or the divider shaft 534, which may enable different medical instruments or devices to be used during a surgical procedure while protecting one or more medical devices or implants from the medical instrument. Moreover, the divider 106, 606 may be coupled to the cannula 104, 604 such that the divider shaft 434 or the divider shaft 534 provides working channels with different volumes. Further, the kit 108 of FIG. 1 or the kit 608 of FIG. 48 may include more than one divider 106, 606 and each divider in the kit 108, 608 may be configured to have a different one of the divider shaft 234, the divider shaft 334, the divider shaft 434, the divider shaft 534 and the divider shaft 734 such that the kit 108, 608 may be used to separate the central lumen 216 into multiple different volumes during a surgical procedure.

In addition, it should be noted that while the suture cleats 162, 662 are described and illustrated herein as being associated with the cannula cap 140, 640, in other examples, the suture cleats 162, 662 may be coupled to, formed on or defined in the cannula 104, 604. For example, one or more suture cleats, such as the suture cleats 162, 662, may be defined in the cannula body 144, 644. In this example, the suture cleats may be defined in the cannula body 144, 644 so as to be on opposed sides of the cannula body 144, 644 and in communication with a respective one of the working channels 270, 272, 370, 372, 470, 472, 570, 572, 574, 770, 772 to receive a portion of the soft anchor 286.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability. In addition, it should be noted herein that the numerical values provided are merely examples, and that the cannula system 100, 600 may be sized to correspond to a predetermined portion of an anatomy for the performance of a surgical procedure.

As used herein, the phrase at least one of A, B, and C should be construed to mean a logical (A OR B OR C), using a non-exclusive logical OR, and should not be construed to mean “at least one of A, at least one of B, and at least one of C.” The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A cannula system, comprising:
a cannula defining a central lumen; and
a divider removably coupled to the cannula, the divider having a divider shaft configured to separate the central lumen into at least two discrete working channels.

2. The cannula system of claim 1, wherein the central lumen is elliptical in cross-section.

3. The cannula system of claim 1, wherein the cannula further comprises a valve received within the central lumen, and the divider shaft extends through the valve.

4. The cannula system of claim 1, wherein the cannula further comprises a cap, and the cap defines at least one suture retaining slot.

5. The cannula system of claim 4, wherein the at least one suture retaining slot defines a tortuous path configured to retain at least one flexible strand.

6. The cannula system of claim 4, wherein the cap further defines a coupling groove configured to receive a portion of the divider, and the coupling groove is spaced apart from the at least one suture retaining slot about a perimeter of the cap.

7. The cannula system of claim 4, wherein the cap defines a central cap bore configured to be in communication with the central lumen.

8. The cannula system of claim 4, wherein the at least one suture retaining slot defines a slit configured to retain at least one flexible strand.

9. The cannula system of claim 1, further comprising an obturator configured to be removably coupled to the central lumen.

10. The cannula system of claim 9, wherein an obturator shaft of the obturator has an elliptical cross-section.

11. The cannula system of claim 1, wherein a center of a first one of the at least two working channels is spaced apart from a second center of a second one of the at least two working channels by at least one millimeter.

12. The cannula system of claim 1, wherein the divider shaft includes at least two side channels and each of the at least two side channels cooperate with a sidewall of the central lumen to define the at least two working channels.

13. A method for soft tissue repair, comprising:
inserting a cannula into an incision in an anatomy, the cannula defining a central lumen;
inserting a divider into the central lumen to separate the central lumen into at least two discrete working channels; and
inserting a soft anchor into one of the at least two working channels to repair soft tissue.

14. The method of claim 13, wherein the central lumen has an elliptical cross-section, and the method further comprises:
orientating the cannula such that a major axis of the cannula is substantially perpendicular to the incision.

15. The method of claim 14, further comprising:
inserting an obturator having an obturator shaft with an elliptical cross-section through the central lumen; and
directing the cannula through the anatomy with the obturator.

16. The method of claim 13, further comprising:
inserting a drill guide into the one of the at least two working channels; and
inserting a drill through the drill guide to form a bone socket prior to inserting the soft anchor.

17. The method of claim 16, further comprising:
tensioning the soft anchor to set the soft anchor within the bone socket;
coupling at least one flexible strand associated with the soft anchor to a suture retaining slot defined in the cannula to couple the at least one flexible strand to the cannula;
inserting the drill guide into a second one of the at least two working channels; and
inserting the drill through the drill guide and the second one of the at least two working channels to form a second bone socket without contacting the at least one flexible strand.

18. The method of claim 17, further comprising:
inserting a second soft anchor into the second one of the at least two working channels;
tensioning the soft anchor to set the soft anchor within the second bone socket; and
coupling at least one second flexible strand associated with the second soft anchor to a second suture retaining slot defined in the cannula to couple the at least one second flexible strand to the cannula.

19. The method of claim 18, further comprising:
coupling the at least one flexible strand of the soft anchor to the second soft anchor;
coupling the at least one second flexible strand of the second soft anchor to the soft anchor;
removing the divider from the cannula; and
tensioning the at least one flexible strand and the at least one second flexible strand to repair the soft tissue.

20. The method of claim 19, further comprising:
removing the cannula from the anatomy after the tensioning the at least one flexible strand and at least one second flexible strand.
